(19) United States
(12) Patent Application Publication
(10) Pub. No.: US 2008/0207536 A1
(43) Pub. Date: Aug. 28, 2008

(54) Antibacterial Agents

(75) Inventors: Biswajit Das, Haryana (IN); Rita Katoch, Chandigarh (IN); Atul Kashinath Hajare, Maharashtra (IN); Gobind Singh Kapkoti, Navi Mumbai (IN); Anish Bandyopadhyay, Haryana (IN); Ramadass Venkataramanan, New Delhi (IN); Rajesh Kumar, Delhi (IN); Santosh Haribhau Kurhade, Maharashtra (IN); Sujata Rathy, Delhi (IN); Anjan Chakrabarti, Haryana (IN); Yogesh Baban Surase, Maharashtra (IN); Mohammad Salman, Princeton, NJ (US); Ashok Rattan, Delhi (IN)

Correspondence Address: RANBAXY INC., 600 COLLEGE ROAD EAST, SUITE 2100, PRINCETON, NJ, 08540, US

(73) Assignee: RANBAXY LABORATORIES LIMITED, Gurgaon, Haryana (IN)

(21) Appl. No.: 11/572,754

(22) PCT Filed: Jul. 13, 2005
(86) PCT No.: PCT/IB2005/001986

§ 371 Date: Jan. 29, 2008

(30) Foreign Application Priority Data
Jul. 28, 2004 (IN) 1399/DEL/2004
May 30, 2005 (IN) 1390/DEL/2005

(51) Int. Cl. A61K 31/7048; C07H 17/08; A61P 31/04

(52) U.S. Cl. 514/29; 536/7.2

(57) ABSTRACT

The present invention provides acylide derivatives, which can be used as antibacterial agents. Compounds disclosed herein can be used for the treatment or prevention of a condition caused by or contributed to by Gram-positive, Gram-negative or anaerobic bacteria, more particularly against bacterium such as Staphylococci, Streptococci, Enterococci, Haemophilus, Moraxalla spp., Chlamydia spp., Mycoplasm, Legionella spp., Myobacterium, Helicobacter, Clostridium, Bacteroides, Corynebacterium, Baccillus or Enterobactericeae. Processes for the preparation of disclosed compounds, pharmaceutical compositions thereof, and method of treating bacterial infections, are also provided.

### FIELD OF THE INVENTION

The present invention provides acylide derivatives, which can be used as antibacterial agents. Compounds disclosed herein can be used for the treatment or prevention of a condition caused by or contributed to by Gram-positive, Gram-negative or anaerobic bacteria, more particularly against bacterium such as Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae. Processes for the preparation of disclosed compounds, pharmaceutical compositions thereof, and method of treating bacterial infections are also provided.

### BACKGROUND OF THE INVENTION

The first generation macrolides erythromycin A and the early derivatives are characterized by bacteriostatic or bactericidal activity for most Gram-positive bacteria, atypical pathogens, and many community acquired respiratory infections and in patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects (Itoh, Z et al., Am. J. Physiol., 247: 688, (1984); Omura, S et al., J. Med. Chem., 30; 1943, (1987). Various erythromycin A derivatives have been prepared to overcome the acid instability and other problems associated with it.

Roxithromycin, clarithromycin and azithromycin have been developed to address limitations of erythromycin A. Both clarithromycin and azithromycin have proved to be important drugs in the treatment and prophylaxis of atypical Mycobacterial infectious in patients with HIV.

Macrolides have proved to be effective drugs in the treatment of many respiratory tract infections. However, increasing resistance among S. pneumoniae has prompted the search for new compounds that retain favorable safety profile, a wide spectrum of activity which is confined to respiratory pathogens. Consequently, investigators have prepared chemical derivatives of erythromycin A in an attempt to obtain analogs having modified or improved profiles of antibiotic activity.

WO 01/10878, 01/10879 and 01/10880 disclose erythromycin derivatives allegedly having potent antibacterial effects on erythromycin-resistant bacteria and Haemophilus influenzae. WO 99/21870 discloses erythromycin A, 11,12-carbamate derivatives described as having antibacterial activity against not only erythromycin-sensitive bacteria but also erythromycin-resistant bacteria. U.S. Pat. No. 5,444,051 discloses erythromycin compounds, antibiotic compositions and a method of combating bacteria infection in warm-blooded animals. U.S. Pat. No. 6,191,118 discloses erythromycin A derivatives having strong antibacterial activity against erythromycin-resistant bacteria. U.S. Pat. No. 5,631,354 discloses 5-O-desosaminylerythronolide derivatives possessing antibacterial activity. U.S. Pat. No. 6,020,521 discloses a class of macrolide compounds, which are antagonists of luteinizing hormone-realising hormone (LHRH).

### SUMMARY OF THE INVENTION

Herein are provided acylide derivatives, which can be used in the treatment or prevention of bacterial infection, and processes for the synthesis of these compounds. Pharmaceutically acceptable salts, pharmaceutically acceptable solvates, stereoisomers, prodrugs, and polymorphs of these compounds having same type of activity are also provided. Pharmaceutical compositions containing the disclosed compounds together with pharmaceutically acceptable carriers, excipients or diluents, which can be used for the treatment of bacterial infection, are also provided.

Other aspects will be set forth in accompanying description which follows and in part will be apparent from the description or may be learnt by the practice of the invention.

In accordance with one aspect, there are provided compounds having the structure of Formula I,

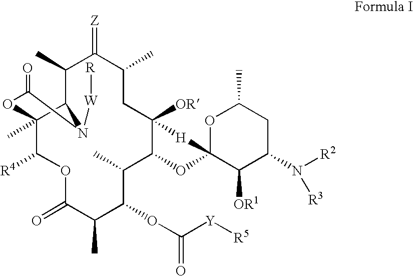

pharmaceutically acceptable salts, pharmaceutically acceptable solvates, stereoisomers, prodrugs or polymorphs thereof, wherein:

R¹ can be hydrogen or a hydroxyl protecting group;

R² and R³ can be independently alkyl, alkenyl, alkynyl, cycloalkyl, aryl, heterocycle, aralkyl or (heterocycle)alkyl (with the proviso that R² and R³ simultaneously can not be methyl);

R⁴ can be alkyl, alkenyl or alkynyl;

R⁵ can be alkyl, aryl or heterocycle;

R can be no atom, hydrogen, aryl or heterocycle;

R′ can be alkyl, —(CH₂)_{q}—U—V (wherein q can be an integer of from 1 to 4, U can be alkenyl or alkynyl, V can be hydrogen, aryl or heterocycle);

W can be alkenyl, -G(CH₂)ₘJ, —CR⁹R¹⁰, —NR⁹— or —SO₂— {[wherein m can be an integer of from 2 to 6, G can be no atom, —CO, —CS, —SO₂ or —NR⁹, J can be no atom, or —N((R⁹)(CH₂)ₙ— (wherein n can be an integer of from 1 to 4, R⁹ and R¹⁰ can be independently hydrogen or alkyl)]}; Y can be -Q(CH₂)ₖ—, {wherein k can be an integer of from 1 to 6, Q can be no atom, —NR⁹— or oxygen [wherein R⁹ can be hydrogen or alkyl]}, further alkylene chain of -Q(CH₂)ₖ— can be optionally substituted with alkyl, hydroxy or alkoxy; and

Z can be oxygen, sulphur or NOR⁸ (wherein R⁸ can be hydrogen, alkyl or aralkyl).

According to one embodiment, compounds of Formula I have the following attributes:

R¹ can be hydrogen; R can be no atom or heterocycle; R² and R³ can be alkyl, alkenyl or alkynyl (however R² and R³ simultaneously cannot be methyl); R⁴ can be ethyl; R⁵ can be alkyl, aryl or heterocycle; R′ and R⁴ can be alkyl; W can be alkenyl or -G(CH₂)ₘJ-; Z can be oxygen or sulphur; Y can be -Q(CH₂)ₖ, wherein G, J, Q, m and k are the same as defined earlier.

In accordance with a second aspect, there is provided a method for treating or preventing a mammal suffering from a condition caused by or contributed to by Gram-positive, Gram-negative or anaerobic bacteria, comprising administering to said mammal a therapeutically effective amount of a compound or a pharmaceutical composition disclosed herein.

The bacterial infection may be caused by bacterium such as Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae.

The conditions may be, for example, community-acquired pneumonia, upper- and lower-respiratory tract infections, skin and soft tissue infections, hospital-acquired lung infections or bone and joint infections, and other bacterial infections such as mastitis, catether infection, foreign body, prosthesis infections or peptic ulcer disease.

In accordance with a third aspect, there are provided processes for the preparation of disclosed compounds.

The following definitions apply to terms as used herein:

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, NHC(═O)NR_{f}R_{q,}, —C(═O)heteroaryl, C(═O)heterocyclyl, —O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents maybe further substituted by 1-3 substituents selected from alkyl, carboxy, —NR_{f}R_{q}, —C(═O)NR_{f}R_{q}, —OC(═O) NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ—{wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents maybe substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “alkylene,” as used herein, refers to —(CH)ₙ— wherein n can be an integer of from 0 to 4 and one or more hydrogen can optionally be substituted with alkyl, hydroxy, halogen or oximes. Alkylene can also be optionally interrupted by —CONH—, —C═O or —C═NOH.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC (═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or SO₂R₆ (wherein R₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (where R₆ is same as defined earlier).

The term “alkynyl,” unless otherwise specified, refers to a monoradical of an unsaturated hydrocarbon, having from 2 to 20 carbon atoms, hi the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, or —SO₂R₆ (where R₆ is same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1] heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC (═O)R_{f}, C(═O)NR_{f}R_{q}, —O—C(═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —OC(═O)NR_{f}R_{q} (wherein R_{f} and R_{q} are the same as defined earlier), cyano or —SO₂R₆ (where R₆ is same as defined earlier). “Cycloalkylalkyl” refers to alkyl-cycloalkyl group linked through alkyl portion, wherein the alkyl and cycloalkyl are the same as defined earlier.

As used herein the term “halogen or halo” refers to fluorine, chlorine, bromine or iodine.

As used herein the term “hydroxyl-protecting group” includes, but are not limited to, acyl, aroyl, alkyl, aryl, butyldiphenylsilyl, methoxymethyl and methylthiomethyl, and the like.

As used herein the term “thio” refers to the group —SH.

As used herein the term “alkoxy” stands for a group O—R wherein R refers to alkyl or cycloalkyl. Examples of alkoxy include, but are not limited to, methoxy, ethoxy, cyclopentoxy and the like.

As used herein the term “thioalkyl” refers to —SR wherein R refers to alkyl or cycloalkyl.

As used herein the term “haloalkyl” refers to alkyl of which one or more hydrogen (s) is/are replaced by halogen.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term ‘heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

As used herein the term “(heterocycle) alkyl” stands for heterocycle, which is bonded to an alkylene chain. Examples of heterocycle alkyl include, but are not limited to, isothiazolidinyl ethyl, isothiazolyl propyl, pyrazinyl methyl, pyrazolinyl propyl and pyridyl butyl, and the like. Aryl and heterocycle groups may optionally be substituted with one or more substituent(s) such as hydroxy, nitro, mercapto, cyano, alkyl, halogen, haloalkyl, alkoxy, thioalkyl, optionally substituted aryl, optionally substituted heterocyclyl, —NR⁶R⁷, —CONR⁶R⁷, —COOR⁷, —CONHR⁷, —OCOR⁷, —COR⁷, —NHSO₂R⁷, and —SO₂NHR⁷, wherein R⁶ and R⁷ can be hydrogen or alkyl.

As used herein the term “polymorphs” includes all crystalline forms and amorphous forms for compounds described herein.

The term “pharmaceutically acceptable solvates” refers to solvates with waters (i.e hydrates) or pharmaceutically acceptable organic solvents. Such solvates are also encompassed within the scope of this invention.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Example of such inorganic acids include, but not limited to, hydrochloric, sulfuric, phosphoric acid, and the like. Appropriate organic acids include, but not limited to, aliphatic, cycloaliphoric, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid, and the like.

“The term pharmaceutically acceptable carriers” is intended to include non-toxic, inert solid, semi-solid or liquid filler, diluent, encapsulating material or formulation auxiliary of any type.

The compounds of present invention include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformational of a reference compound that converts to the reference compound only slowly on the NMR or laboratory time scale. Conformational isomers (or conformers or rotational isomers or rotamers) are stereoisomers produced by rotation about σ bonds, and are often rapidly interconverting at room temperature. Racemic mixtures are also encompassed within the scope of this invention.

The present invention also includes within its scope prodrugs of these agents. In general, such “prodrugs” will be functional derivatives of these compounds, which are readily convertible in vivo into the required compound. Conventional procedures for the selection and preparation of suitable prodrug derivatives are described, for example, in “Design of Prodrugs”, ed. H Bundgaard, Elsevier, 1985.

### DETAILED DESCRIPTION OF THE INVENTION

The compounds provided herein may be prepared by techniques well known in the art and familiar to the average synthetic organic chemist. In addition, the compounds of the present invention may be prepared by following reaction sequences such as those depicted in Schemes I, II and III.

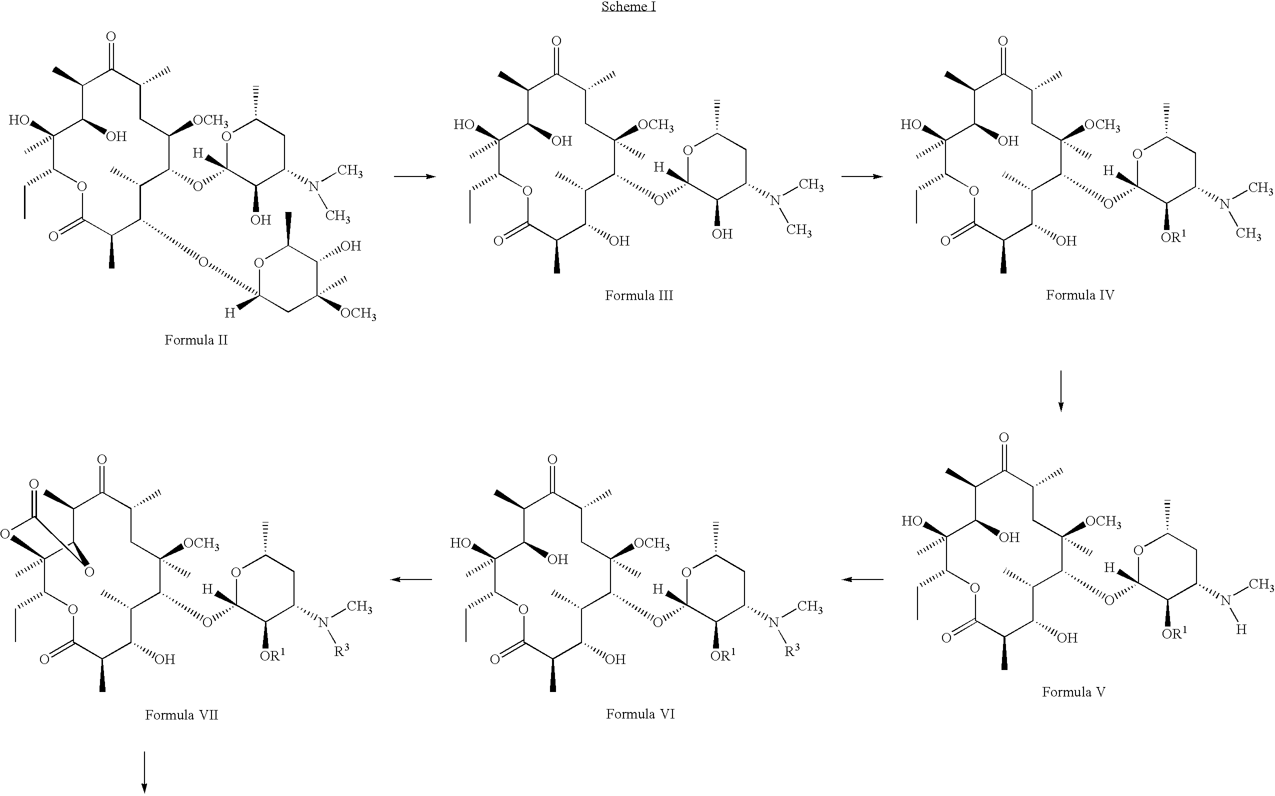

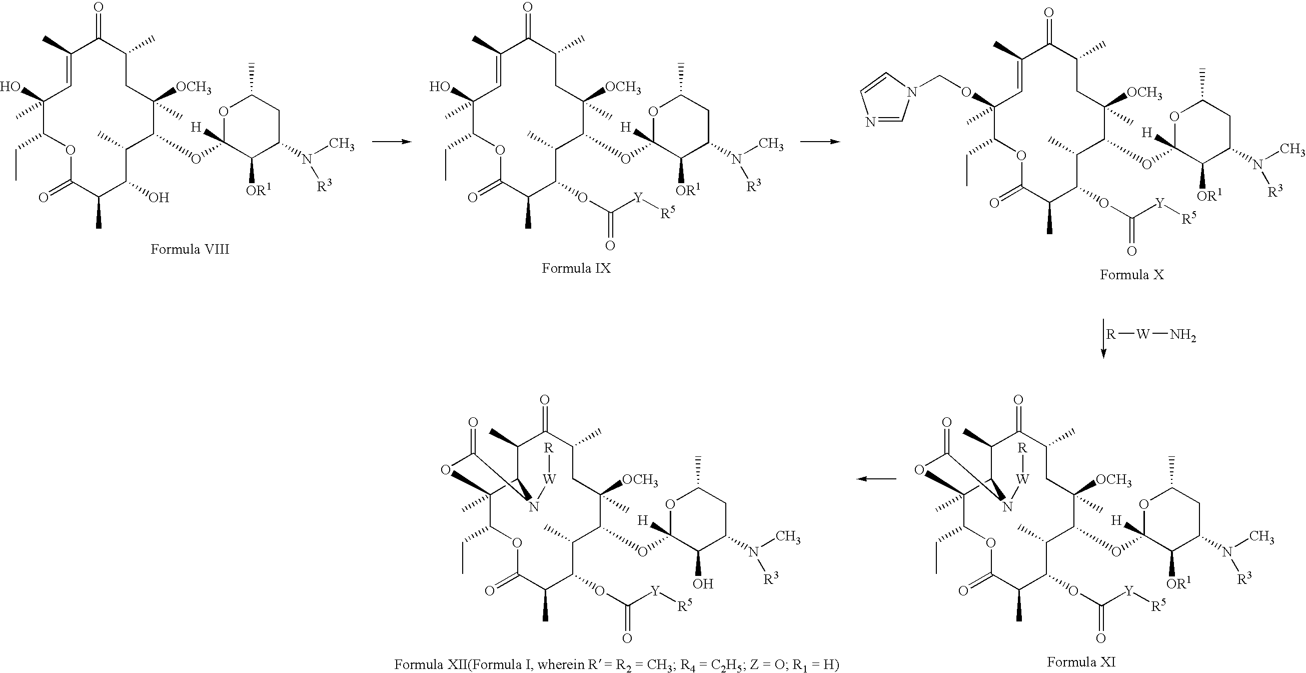

The compound of Formula XII can be prepared according to Scheme I. Thus, clarithromycin of Formula II is hydrolyzed to give a compound of Formula III, which on protection with a reagent of Formula R¹₂O or R¹X (wherein X is halogen) gives a compound of Formula IV (wherein R¹ is —COPh), which on desmethylation at 3′-N-dimethyl group gives a compound of Formula V, which on alkylation with a reagent of Formula R³CHO, R³₂CO or R³X gives a compound of Formula VI (wherein R³ is the same as defined earlier), which on reaction with a suitable reagent gives a compound of Formula VII, which on reaction with a suitable organic base gives a compound of Formula VIII, which on acylation with a reagent of Formula R⁵YCOOH, (R⁵YCO)₂O, R⁵YCOX or R⁵YCOOR¹⁰ (wherein R¹⁰ is leaving group such as pivaloyl, p-toleuensulfonyl, isobutoxycarbonyl, ethoxycarbonyl or isopropoxycarbonyl) gives a compound of Formula IX (wherein Y and R⁵ are the same as defined earlier), which on reaction with N,N′-carbonyl diimidazole gives a compound of Formula X, which on reaction with a compound of Formula R—W—NH₂ gives a compound of Formula XI (wherein R and W are the same as defined earlier), which is finally deprotected to give a compound of Formula XII.

The hydrolysis of clarithromycin of Formula II to give a compound of Formula III can be carried out in the presence of an inorganic or organic acid, for example, hydrochloric acid, sulphuric acid or dichloroacetic acid.

The hydroxyl protection of a compound of Formula III to give a compound of

Formula IV can be carried out in a solvent, for example, dichloromethane, dichloroethane, chloroform or ethyl acetate. The hydroxyl protection of a compound of Formula III to give a compound of Formula IV can be carried out in the presence of an organic base, for example, triethylamine, pyridine, tributylamine or 4-N-dimethylaminopyridine.

The desmethylation of a compound of Formula IV to give a compound of Formula V can be carried out in the presence of a demethylating agent, for example, N-iodosuccinamide or diisopropyl azodicarboxylate. The desmethylation of a compound of Formula IV can be carried out in a solvent, for example, acetonitrile, tetrahydrofuran, dichloromethane, dichloroethane, ethyl acetate or mixture thereof. The quenching of desmethylation reaction can be carried out in the presence of a quenching agent, for example, sodium bisulphite, sodium carbonate or mixture thereof.

The alkylation of a compound of Formula V to give a compound of Formula VI can be carried out in a solvent, for example, dimethylformamide, acetonitrile or tetrahydrofuran. The alkylation of a compound of Formula V to give a compound of Formula VI can be carried out in an inorganic or organic base, for example, sodium hydrogen carbonate, potassium carbonate, sodium hydride, pyridine, triethylamine or diisopropyl ethylamine. The alkylation of a compound of Formula V can also be carried out with a reagent of Formula R³CHO with a reducing agent, for example, sodium cyanoborohydride, sodium borohydride or sodium triacetoxyborohydride in the presence of an organic acid, for example, acetic acid or dichloroacetic acid in a solvent, for example, methanol, ethanol, propanol or isopropanol.

The reaction of a compound of Formula VI to give a compound of Formula VII can be carried out in the presence of a reagent, for example, triphosgene or ethylene dicarbonate. The reaction of a compound of Formula VI to give a compound of Formula VII can be carried out in a solvent, for example, chloroform, dichloromethane, carbon tetrachloride or dichloroethane. The reaction of a compound of Formula VI can be carried out in the presence of an organic base, for example, triethylamine, pyridine, tributylamine or 4-N-dimethylaminopyridine.

The reaction of a compound of Formula VII to give a compound of Formula VIII can be carried out in a solvent, for example, dimethylformamide, tetrahydrofuran or dimethylsulphoxide. The reaction of a compound of Formula VII to give a compound of Formula VIII can be carried out in the presence of an organic base, for example, tetramethyl guanidine, pyridine or trimethylamine.

The reaction of a compound of Formula VIII to give a compound IX can be carried out in a solvent, for example, dichloromethane, dichloroethane, acetone, ethyl acetate or tetrahydrofuran. The reaction of a compound of Formula VIII to give a compound IX can be carried out in the presence of an inorganic or organic base, for example, sodium bicarbonate, potassium carbonate, triethylamine, pyridine, tributylamine or 4-N-dimethylaminopyridine. The reaction of a compound of Formula VIII to give a compound IX can be carried out in the presence of an activating agent, for example, dicyclohexylcarbodiimide or 1-ethyl-3-(3-dimethylaminopropyl) carbodiimide hydrochloride.

The reaction of a compound of Formula IX with N,N′-carbonyl diimidazole to give a compound of Formula X can be carried out in a solvent, for example, dimethylformamide, tetrahydrofuran or mixture thereof. The reaction of a compound of Formula IX can be carried out in the presence of an inorganic base, for example, sodium hydrogen carbonate, potassium carbonate or sodium hydride.

The reaction of a compound of Formula X with a compound of Formula R—W—NH₂ to give a compound of Formula XI can be carried out in a solvent, for example, acetonitrile, water, dimethylformamide or mixture thereof.

The deprotection of a compound of Formula XI to give a compound of Formula XII can be carried out in an alcohol, for example, methanol, ethanol, propanol or isopropanol.

The compound of Formula XII can further be converted into its salt by following a conventional method, for example, those known in the prior art.

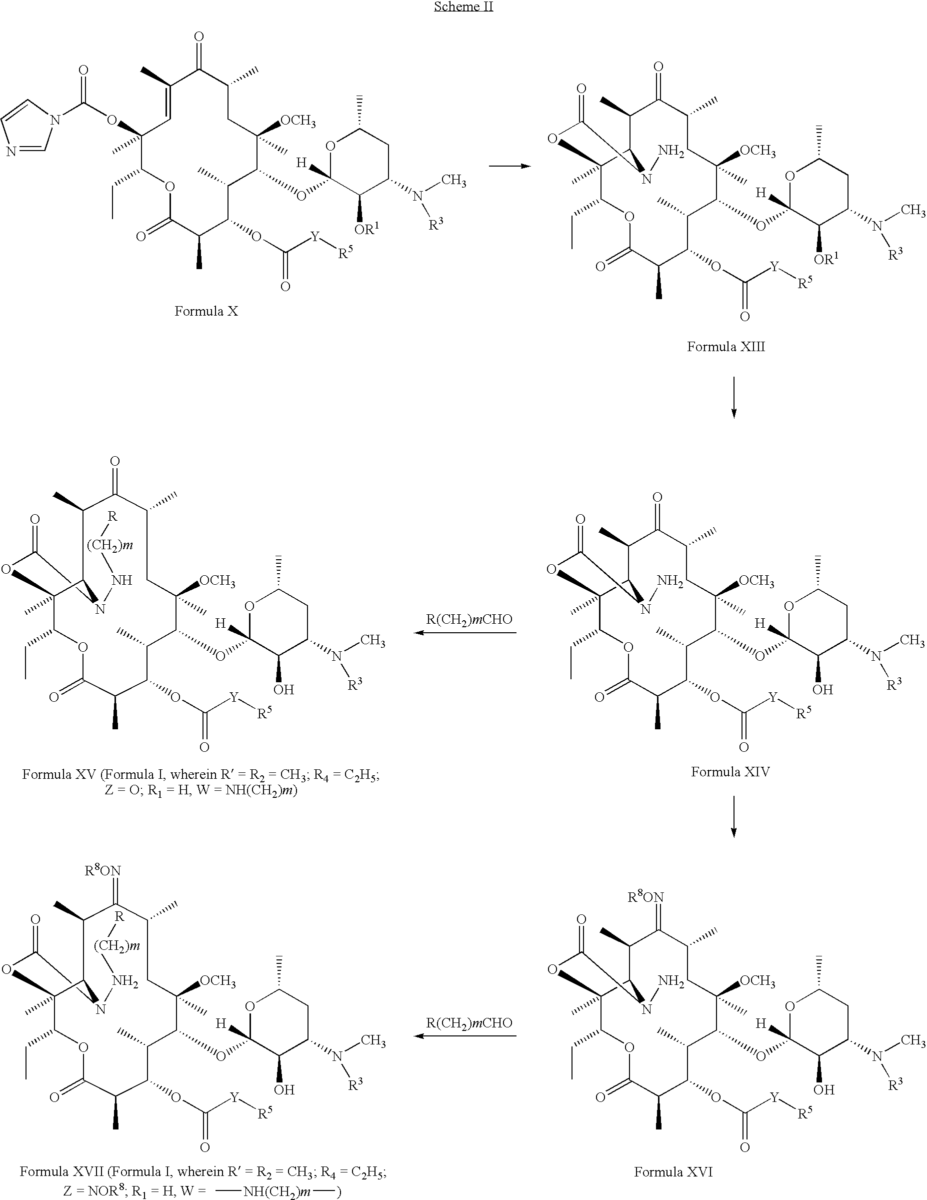

The compounds of Formula XV and XVII can be prepared according to Scheme II. Thus, a compound of Formula X (from scheme I) is reacted with hydrazine hydrate to give a compound of Formula XIII, which on deprotection gives a compound of Formula XIV,
(a) which is finally reacted with a compound of Formula R (CH₂)ₘCHO to give a compound of Formula XV (wherein R and m are the same as defined earlier), or
(b) which on reaction with a compound of Formula R₈ONH₂.hydrochloride gives a compound of Formula XVI (wherein R₈ is the same as defined earlier), which is finally reacted with a compound of Formula R (CH₂)ₘCHO to give a compound of Formula XVII (wherein R and m are the same as defined earlier).

The reaction of a compound of Formula X with hydrazine hydrate to give a compound of Formula XIII can be carried out in a solvent, for example, dimethylformamide, tetrahydrofuran or dimethylsulphoxide.

The deprotection of a compound of Formula XIII to give a compound of Formula XIV can be carried out in a solvent, for example, methanol, ethanol, propanol or isopropanol.

The reaction of a compound of Formula XIV with a compound of Formula R(CH₂)ₘCHO to give a compound of Formula XV can be carried out in a solvent, for example, methanol, ethanol, propanol or isopropanol. The reaction of a compound of Formula XIV to give a compound of Formula XV can be carried out in the presence of an organic acid, for example, acetic acid or dichloroacetic acid. The reaction of a compound of Formula XIV to give a compound of Formula XV can be carried out in the presence of a reducing agent, for example, sodium borohydride, sodium cyanoborohydride or sodium triacetoxyborohydride.

The reaction of a compound of Formula XIV with a compound of Formula R₈ONH₂.hydrochloride to give a compound of Formula XVI can be carried out in a solvent, for example, methanol, ethanol, propanol or isopropanol.

The reaction of a compound of Formula XVI with a compound of Formula R(CH₂)ₘCHO to give a compound of Formula XVII can be carried out in a solvent, for example, methanol, ethanol, propanol or isopropanol. The reaction of a compound of Formula XVI to give a compound of Formula XVII can be carried out in the presence of a reducing agent, for example, sodium borohydride, sodium cyanoborohydride or sodium triacetoxyborohydride.

The compounds of Formula XV and XVII can further be converted into their salt by following a conventional method, for example those known in the prior art.

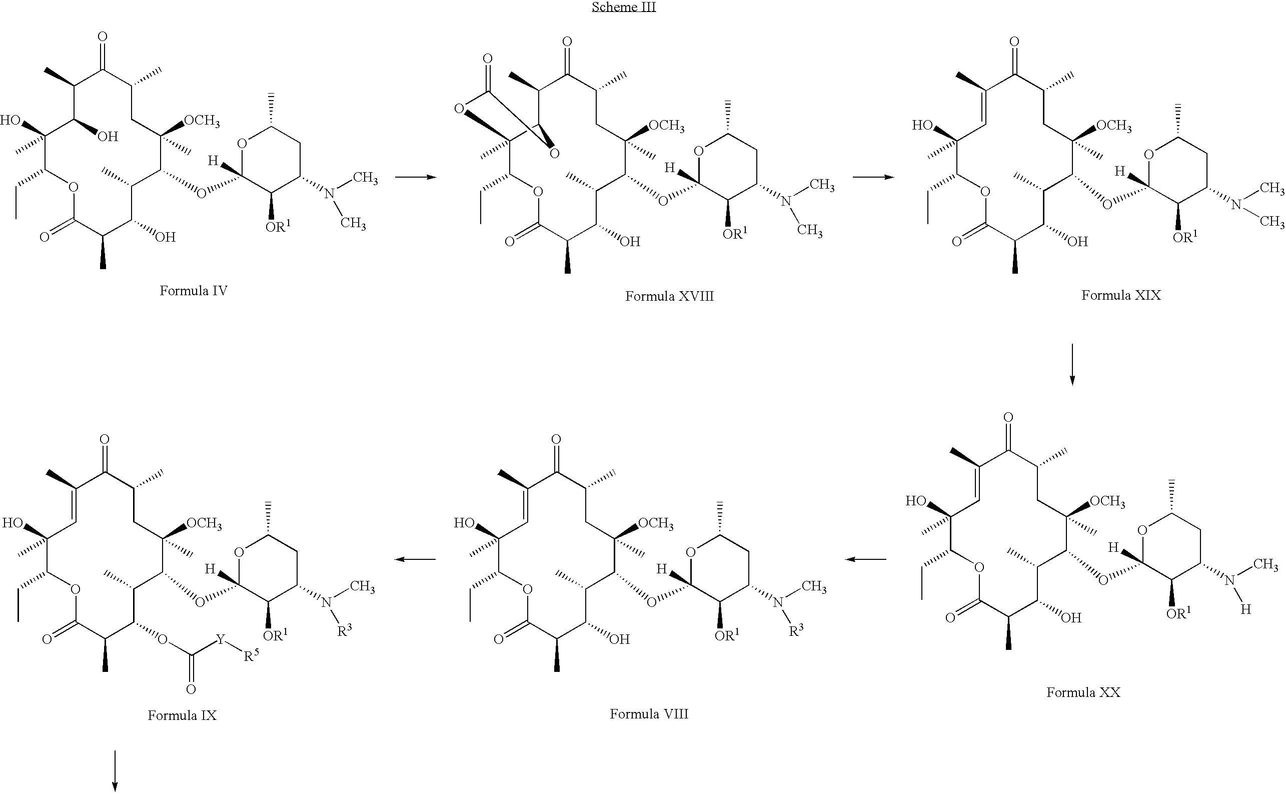

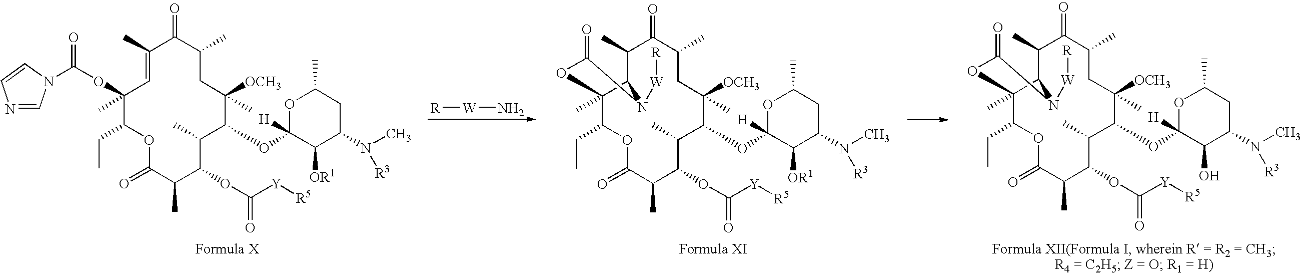

A compound of Formula XII can also be prepared according to Scheme III. Thus, reaction of a compound of Formula IV (wherein R¹ is —COPh) with a reagent gives a compound of Formula XVIII, which on reaction with an organic base gives a compound of Formula XIX, which on desmethylation at 3′-N-dimethyl group gives a compound of Formula XX, which on alkylation with a reagent of Formula R³CHO, R³₂CO or R³X gives a compound of Formula VIII (wherein R³ is the same as defined earlier), which on acylation with a reagent of Formula R⁵YCOOH, (R⁵YCO)₂O, R⁵YCOX or R⁵YCOOR^{10 (wherein R}¹⁰ is leaving group such as pivaloyl, p-toleuensulfonyl, isobutoxycarbonyl, ethoxycarbonyl or isopropoxycarbonyl) gives a compound of Formula IX (wherein Y and R⁵ are the same as defined earlier), which on reaction with N,N′-carbonyl diimidazole gives a compound of Formula X, which on reaction with a compound of Formula R—W—NH₂ gives a compound of Formula XI (wherein R and W are the same as defined earlier), which is finally deprotected to give a compound of Formula XII.

The reaction of a compound of Formula IV to give a compound of Formula XVIII can be carried out in the presence of a reagent, for example, triphosgene or ethylene carbonate. The reaction of a compound of Formula IV to give a compound of Formula XVIII can be carried out in a solvent, for example, chloroform, dichloromethane, carbon tetrachloride or dichloroethane. The reaction of a compound of Formula IV can be carried out in the presence of an organic base, for example, triethylamine, pyridine, tributylamine, 4-N-dimethylaminopyridine or diisopropyl ethyl amine.

The reaction of a compound of Formula XVIII to give a compound of Formula XIX can be carried out in a solvent, for example, dimethylformamide, tetrahydrofuran or dimethylsulphoxide. The reaction of a compound of Formula XVIII to give a compound of Formula XIX can be carried out in the presence of an organic base, for example, tetramethyl guanidine, pyridine, trimethylamine or diisopropyl ethyl amine.

The desmethylation of a compound of Formula XIX to give a compound of Formula XX can be carried out in the presence of a demethylating agent, for example, N-iodosuccinamide, iodine in acetic acid or diisopropyl azodicarboxylate. The desmethylation of a compound of Formula XIX can be carried out in a solvent, for example, acetonitrile, tetrahydrofuran, dichloromethane, dichloroethane, chloroform, carbon tetrachloride, ethyl acetate or mixture thereof. The quenching of desmethylation reaction can be carried out in the presence of a quenching agent, for example, sodium bisulphite, potassium carbonate, sodium acetate, sodium carbonate or mixture thereof.

The alkylation of a compound of Formula XX with a reagent of Formula R³X to give a compound of Formula VIII can be carried out in a solvent, for example, dimethylformamide, acetonitrile or tetrahydrofuran. The alkylation of a compound of Formula XX to give a compound of Formula VIII can be carried out in an inorganic or organic base, for example, sodium hydrogen carbonate, potassium carbonate, sodium acetate, sodium thiosulfate, sodium hydride, pyridine, triethylamine or diisopropyl ethyl amine. The alkylation of a compound of Formula XX can also be carried out with a reagent of Formula R³CHO or R³₂O with a reducing agent, for example, sodium cyanoborohydride, sodium borohydride or sodium triacetoxyborohydride in the presence of an organic acid, for example, acetic acid or dichloroacetic acid in a solvent, for example, methanol, ethanol, propanol or isopropanol.

The reaction of a compound of Formula VIII to give a compound IX can be carried out in a solvent, for example, dichloromethane, dichloroethane, acetone, ethyl acetate or tetrahydrofuran. The reaction of a compound of Formula VIII to give a compound IX can be carried out in the presence of an inorganic or organic base, for example, sodium bicarbonate, potassium carbonate, triethylamine, pyridine, tributylamine or 4-N-dimethylaminopyridine. The reaction of a compound of Formula VIII to give a compound IX can be carried out in the presence of an activating agent, for example, dicyclohexylcarbodiimide or 1-ethyl-3-(3-dimethylaminopropyl) carbodiimide hydrochloride.

The reaction of a compound of Formula IX with N,N′-carbonyl diimidazole to give a compound of Formula X can be carried out in a solvent, for example, dimethylformamide, tetrahydrofuran or mixture thereof. The reaction of a compound of Formula IX can be carried out in the presence of an inorganic base, for example, sodium hydrogen carbonate, potassium carbonate or sodium hydride.

The reaction of a compound of Formula X with a compound of Formula R—W—NH₂ to give a compound of Formula XI can be carried out in a solvent, for example, acetonitrile, water, dimethylformamide or mixture thereof.

The deprotection of a compound of Formula XI to give a compound of Formula XII can be carried out in an alcohol, for example, methanol, ethanol, propanol or isopropanol.

The compound of Formula XII can further be converted into its salt by following a conventional method, for example those known in the prior art.

In the above schemes, where the specific bases, activating agents, solvents, etc., are mentioned, it is to be understood that bases, activating agents, solvents, etc., known to those skilled in the art may be used. Similarly, the reaction temperature and duration may be adjusted according to the desired needs. The compounds provided, or their intermediates can be converted to epimers during the course of reaction and such epimers are also encompassed within the scope of this invention.

The compounds disclosed herein possess antibacterial activity against Gram-positive, Gram-negative and anaerobic bacteria. They are useful as antibacterial agents for the treatment of bacterial infections in human and animal. Compounds provided herein which are useful for such purposes are listed below:
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((3-imidazol-1-yl)-propyl)-imino]erythromycin A (Compound No. 1),
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-imidazol-1-yl)-butyl)-imino]erythromycin A (Compound No. 2),
11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-((3-imidazol-1-yl)-propyl)-imino]erythromycin A (Compound No. 3),
11,12-Dideoxy-3-O-decladinosyl-3-O-(2-nitrophenylacetyl)-5-O-(3′-N-desmethyl-3′-N-ethyl)-6-O-methyl-12,11-[oxycarbonyl-((4-(3H)-imidazo(4,5-b)-pyridin-3-yl)-butyl)-imino]erythromycin A (Compound No. 4),
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-benzimidazol-1-yl)-butyl)-imino]erythromycin A (Compound No. 5),
11,12-Dideoxy-3-O-decladinosyl-3-O-(3-nitrophenyl acetyl-5-O-(3′-N-desmethyl-3′-N-allyl)-6-O-methyl-12,11-[oxycarbonyl-((4-benzimidazol-1-yl)-butyl)-imino]erythromycin A (Compound No. 6),
11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-(prop-2-en-yl)-imino]erythromycin A (Compound No. 7),
11,12-Dideoxy-3-O-decladinosyl-3-O-(4-pyridyl acetyl)-5-O-(3′-N-desmethyl-3′-N-ethyl)-6-O-methyl-12,11-[oxycarbonyl-((4-imidazol-1-yl)-butyl)-imino]erythromycin A (Compound No. 8),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-imidazol-1-yl)-butyl)-imino]erythromycin A (Compound No. 9),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A (Compound No. 10),
11,12-Dideoxy-3-O-decladinosyl-3-O-(4-pyridylacetyl)-5-O-(3′-N-desmethyl-3′-N-ethyl)-6-O-methyl-12,11-[oxycarbonyl-((4-benzimidazol-1-yl)-butyl)-imino]erythromycin A (Compound No. 11),
11,12-Dideoxy-3-O-decladinosyl-3-O-(2-pyridylacetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A (Compound No. 12),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A (Compound No. 13),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A (Compound No. 14),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A (Compound No. 15),
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A (Compound No. 16),
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A (Compound No. 17),
11,12-Dideoxy-3-O-decladinosyl-3-O-(3pyridyl acetyl)-5-O-(3′-N-desmethyl-3′-N-ethyl)-6-O-methyl-12,11-[oxycarbonyl-((4-indol-1-yl)-butyl)-imino]erythromycin A (Compound No. 18),
11,12-Dideoxy-3-O-decladinosyl-3-O-(3-pyridyl acetyl-5-O-(3′-N-desmethyl-3′-N-ethyl)-6-O-methyl-12,11-[oxycarbonyl-((4-benzimidazol-1-yl)-butyl)-imino]erythromycin A (Compound No. 19),
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A (Compound No. 20),
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A (Compound No. 21),
11,12-Dideoxy-3-O-decladinosyl-3-O-(3-pyridylacetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A (Compound No. 22),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-4-ylmethyl)-2-aminoethyl)-imino]erythromycin A (Compound No. 23),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A (Compound No. 24),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-3-ylmethyl)-2-aminoethyl)-imino]erythromycin A (Compound No. 25),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A (Compound No. 26),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A (Compound No. 27),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A (Compound No. 28),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A (Compound No. 29),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-(2-methyl)-benzoimidazo-1-yl)-butyl)-imino]erythromycin A (Compound No. 30),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A (Compound No. 31),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-pyridin-2-yl)-butyl)-imino]erythromycin A (Compound No. 32),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-pyridin-4-yl)-butyl)-imino]erythromycin A (Compound No. 33),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-benzoimidazo-1-yl)-butyl)-imino]erythromycin A (Compound No. 34),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A (Compound No. 35),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-benzotriazol-1-yl)-butyl)-imino]erythromycin A (Compound No. 36),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A (Compound No. 37),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A (Compound No. 38),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A (Compound No. 39),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-pyrrolo[2,3-b]pyridin-1-yl)-butyl)-imino]erythromycin A (Compound No. 40),
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-(3H)imidazo[4,5-b]pyridin-3-yl)-butyl)-imino]erythromycin A (Compound No. 41),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A (Compound No. 42),
11,12-dideoxy-3-O-decladinosyl-3-O-(2-pyridylacetyl)-5-O-(3′-N-desmethyl-3′-N-allyl)-6-O-methyl-12,11-[oxycarbonyl-((9-(4-amino-butyl)9H-purin-6-yl)-imino]erythromycin A (Compound No. 43),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-benzoimidazo-1-yl)-butyl)-imino]erythromycin A (Compound No. 44),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A (Compound No. 45),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A (Compound No. 46),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A (Compound No. 47),
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 48),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A (Compound No. 49),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A (Compound No. 50),
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-benzoimidazo-1-yl)-butyl)-imino]erythromycin A (Compound No. 51),
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 52),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A (Compound No. 53),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-benzotriazol-1-yl)-butyl)-imino]erythromycin A (Compound No. 54),
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A (Compound No. 55),
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-pyrrolo[2,3-b]pyridin-1-yl)-butyl)-imino]erythromycin A (Compound No. 56),
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-furan-2-yl)-imidazol-1-yl)-butyl)-imino]erythromycin A (Compound No. 57),
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-thiophen-2-yl)-imidazol-1-yl)-butyl)-imino]erythromycin A (Compound No. 58),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3[4-(6-amino-9H-purin-9-yl)propyl)hydrazo)]erythromycin A (Compound No. 59),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(quinolin-8-yl)-butyl)-imino)]erythromycin A (Compound No. 60),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-8-yl)-butyl)-imino)]erythromycin A (Compound No. 61),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-4-yl)-butyl)-imino)]erythromycin A (Compound No. 62),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1-methyl-1H-pyrrolo[2,3-b]pyridin-3-yl)-butyl)-imino)]erythromycin A (Compound No. 63),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-amino-9H-purin-9-yl butylimino)]erythromycin A (Compound No. 64),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A (Compound No. 65),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A (Compound No. 66),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A (Compound No. 67),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A (Compound No. 68),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A hydrochloride salt (Compound No. 69)
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A (Compound No. 70),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(6-pyrrol-1-yl-purin-9-yl)-butyl)-imino)]erythromycin A (Compound No. 71),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-diethylamino-9H-purin-9-yl butylimino)]erythromycin A (Compound No. 72),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-ethylamino-9H-purin-9-yl butylimino)]erythromycin A (Compound No. 73),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A (Compound No. 74),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A (Compound No. 75),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A (Compound No. 76),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A (Compound No. 77),
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A (Compound No. 78),
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A (Compound No. 79),
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A (Compound No. 80),
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A (Compound No. 81),
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A (Compound No. 82),
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A (Compound No. 83),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A (Compound No. 84),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-methylamino-9H-purin-9-yl butylamino)]erythromycin A (Compound No. 85),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(purin-9-yl)-butyl)-imino)]erythromycin A (Compound No. 86),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(purin-9-yl)-butyl)-imino)]erythromycin A (Compound No. 87),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-c]pyridin-1-yl)-butyl)-imino)]erythromycin A (Compound No. 88),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(3H-imidazo[4,5-c]pyridin-3-yl)-butyl)-imino)]erythromycin A (Compound No. 89),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-1yl)-butyl)-imino)]erythromycin A (Compound No. 90),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A (Compound No. 91),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazol[4,5-c]pyridin-3-yl)-butyl)-imino)]erythromycin A (Compound No. 92),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A (Compound No. 93),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-oxazol-5-yl-1H-imidazol-1yl)-butyl)-butyl)-imino)]erythromycin A (Compound No. 94),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A (Compound No. 95),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A hydrochloride salt (Compound No. 96),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A (Compound No. 97),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A (Compound No. 98),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-2-yl)-pyrazol-1-yl)-butylimino)]erythromycin A (Compound No. 99),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-2-yl)-pyrazol-1-yl)-butylimino)]erythromycin A (Compound No. 100),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-2-yl)-pyrazol-1-yl)-butylimino)]erythromycin A (Compound No. 101),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-2-yl)-1H-pyrazol-1-yl)-butylimino)]erythromycin A (Compound No. 102),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A (Compound No. 103),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-(quinolin-3-yl)-butyl)-imino)]erythromycin A (Compound No. 104),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-3-yl)-butyl)-imino)]erythromycin A (Compound No. 105),
11,12-dideoxy-3-O-decladinosyl-3-O-(2-pyridyl acetyl)-5-O-(3′-N-desmethyl-3′-N-allyl)-6-O-methyl-12,11-[oxycarbonyl-4-([1,4′]-bipyrazol-1′-yl)-butylimino)]erythromycin A (Compound No. 106),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-thiazolyl)-imidazol-1-yl)-butylimino)]erythromycin A (Compound No. 107),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiazol-2-yl)-imidazol-1-yl)-butylimino)]erythromycin A (Compound No. 108),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A (Compound No. 109),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A (Compound No. 110),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A (Compound No. 111),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A (Compound No. 112),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A (Compound No. 113),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-benzoimidazol-1-yl)-propyl)-hydrazo)]erythromycin A (Compound No. 114),
(R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1yl)-propyl)-hydrazo)]erythromycin A (Compound No. 115),
(S or R) 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A (Compound No. 116),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(3-(4-pyridin-3-yl-1H-imidazol-1-yl)-propyl)-hydrazo)]erythromycin A (Compound No. 117),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-pyridin-3-yl-1H-imidazol-1yl)-propyl)-hydrazo)]erythromycin A (Compound No. 118),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(4-pyridin-3-yl-1H-imidazol-1yl)-propyl)-hydrazo)]erythromycin A (Compound No. 119),
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(3-(6-amino-9H-purin-9-yl-propyl)hydrazo)]erythromycin A (Compound No. 120),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1yl)-propyl)-hydrazo)]erythromycin A (Compound No. 121),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A (Compound No. 122),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A (Compound No. 123),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A (Compound No. 124),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-2-yl)-1H-imidazol-1-yl)propyl)-hydrazo)]erythromycin A (Compound No. 125),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A (Compound No. 126),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A (Compound No. 127),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A (Compound No. 128),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A (Compound No. 129),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A (Compound No. 130),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A (Compound No. 131),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-3-yl-1H-imidazol-1-yl)-propyl)-hydrazo)]erythromycin A (Compound No. 132),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A (Compound No. 133),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A (Compound No. 134),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A (Compound No. 135),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A (Compound No. 136),
(R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A (Compound No. 137),
(S or R) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A (Compound No. 138),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-hydrazo)]erythromycin A (Compound No. 139),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A, 9-(O-methyl)oxime (Compound No. 140),
11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A, 9-(O-methyl)oxime (Compound No. 141),
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-(6-fluoro-pyridin-3-yl)-1H-imidazol-1-yl)-butyl)-imino)]erythromycin A (Compound No. 142),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(6-fluoro-pyridin-3-yl)-1H-imidazol-1-yl)-butyl)-imino)]erythromycin A (Compound No. 143),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(1H-imidazol-1-yl)phenyl)-butyl)-imino)]erythromycin A (Compound No. 144),
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(5-(3-aminophenyl)-thiazol-2-yl)-butyl)-imino)]erythromycin A (Compound No. 145),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-amino-pyrimidin)-5-yl)-imidazol-1-yl)-butylimino)]erythromycin A (Compound No. 146),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-aminopyridin-4-yl)-imidazol-1-yl)-butylimino)]erythromycin A (Compound No. 147),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-aminopyridin-3-yl)-imidazol-1-yl)-butylimino)]erythromycin A (Compound No. 148),
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-(dimethylamino)-9H-purin-9-yl)-butylimino)]erythromycin A (Compound No. 149),
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(3-(5-phenyl-tetrazol-1-yl)propylimino)]erythromycin A (Compound No. 150),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyridin-3-yl-pyrazol-1-yl)-butylimino)]erythromycin A (Compound No. 151),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methyl-2H-tetrazol-5-yl)phenyl)butylimino)]erythromycin A (Compound No. 152),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(3-thienyl)-imidazol-1-yl)butylimino)]erythromycin A (Compound No. 153),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(5-methyl-[1,3,4]-oxadiazol-2-yl)phenyl)butylimino)]erythromycin A (Compound No. 154),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([2,3′]-bithiophenyl-5-yl)butylimino)]erythromycin A (Compound No. 155),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([2,3′]-bithiophenyl5-yl)butylimino)]erythromycin A (Compound No. 156),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A (Compound No. 157),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A (Compound No. 158),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A (Compound No. 159),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-imidazol-1-yl)butylimino)]erythromycin A (Compound No. 160),
11,12-dideoxy-3-O-decladinosyl-3-O-(2-methylpropanoyl)-5-O-(3′-N-desmethyl-3′-N-allyl)-6-O-methyl-12,11-[oxycarbonyl4-(4-(4-(furan-2-yl)-imidazol-1-yl)-butylimino)]erythromycin A (Compound No. 161),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(4-methoxy-phenyl)-imidazol-1-yl)-butylimino)]erythromycin A (Compound No. 162),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(4-methoxy-phenyl)-imidazol-1-yl)-butylimino)]erythromycin A (Compound No. 163),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A (Compound No. 164),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H-imidazol-1-yl)-butylimino)]erythromycin A (Compound No. 165),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A (Compound No. 166),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A (Compound No. 167),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2-yl)butylimino)]erythromycin A (Compound No. 168),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A (Compound No. 169),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A (Compound No. 170),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A (Compound No. 171),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A (Compound No. 172),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A (Compound No. 173),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A (Compound No. 174),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A (Compound No. 175),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl)butylimino)]erythromycin A (Compound No. 176),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3((3-(4-(imidazol-1-yl)-pyrazol-1-yl)-propyl)-hydrazo)]erythromycin A (Compound No. 177),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrazol-1-yl]-imidazol-1-yl)-propyl)-hydrazo)]erythromycin A (Compound No. 178),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A (Compound No. 179),
11,12-dideoxy-3-O-decladinosyl-3-O-(3-pyridyl acetyl)-5-O-(3′-N-desmethyl-3′-N-allyl)-6-O-methyl-12,11-[oxycarbonyl-3-((3-([1,4′]-bipyrazol-1′-yl)-propyl)-hydrazo)]erythromycin A (Compound No. 180),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A (Compound No. 181),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A (Compound No. 182),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrimidin-2-yl-imidazol-1-yl)-propyl)-hydrazo]erythromycin A (Compound No. 183),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A (Compound No. 184),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A (Compound No. 185),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(thiophen-2-yl)-tetrazol-2-yl])-butylimino)]erythromycin A (Compound No. 186),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-1-yl]-butylimino)]erythromycin A (Compound No. 187),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-1-yl]-butylimino)]erythromycin A (Compound No. 188),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A (Compound No. 189),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-3-yl-tetrazol-2-yl)-butylimino)]erythromycin A (Compound No. 190),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A (Compound No. 191),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A (Compound No. 192),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A (Compound No. 193),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A (Compound No. 194),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(thiophen-2-yl-tetrazol-2-yl)-butylimino)]erythromycin A (Compound No. 195),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A (Compound No. 196),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A (Compound No. 197),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl )-butylimino)]erythromycin A (Compound No. 198),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A (Compound No. 199),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A (Compound No. 200),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A (Compound No. 201),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A (Compound No. 202),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A (Compound No. 203),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(benzimidazol-1-yl)-butylimino)]erythromycin A (Compound No. 204),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-(3-pyridyl)-imidazol-1-yl)-butylimino)]erythromycin A (Compound No. 205),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A (Compound No. 206),
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(6-amino-9H-purin-9-yl)butylimino)]erythromycin A (Compound No. 207),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-bipyrazol-1′-yl)butylimino]erythromycin A (Compound No. 208),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A (Compound No. 209),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl) butylimino)]erythromycin A (Compound No. 210),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A (Compound No. 211),
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A (Compound No. 212),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A (Compound No. 213),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A (Compound No. 214),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A (Compound No. 215),
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(4-(6-fluoro-pyridin-3-yl)-imidazol-1-yl)butylimino)]erythromycin A (Compound No. 216),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A (Compound No. 217),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A (Compound No. 218),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-((3-(4-pyrimidin-2-yl)-imidazol-2-yl)propyl)-hydrazo)]erythromycin A (Compound No. 219),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(4-pyrimidin-5-yl)-imidazol-2-yl)propyl)-hydrazo)]erythromycin A (Compound No. 220),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A (Compound No. 221),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A (Compound No. 222),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-phenyl)butylimino)]erythromycin A (Compound No. 223),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrimidin-5-yl-imidazol-1-yl)propyl)hydrazo)]erythromycin A (Compound No. 224),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(imidazol-1-yl)-pyrazol-1-yl]propyl}hydrazo)]erythromycin A (Compound No. 225),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-{[3-([1,4′]-biimidazol-1′-yl)propyl}hydrazo)]erythromycin A (Compound No. 226),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A (Compound No. 227),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A (Compound No. 228),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A (Compound No. 229),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A (Compound No. 230),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A (Compound No. 231),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2H-tetrazol-5-yl)-phenyl)butylimino)]erythromycin A (Compound No. 232),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(2H-tetrazol-5-yl)-phenyl]butylimino)]erythromycin A (Compound No. 233),
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A (Compound No. 234),
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A (Compound No. 235),
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A (Compound No. 236),
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-fluoro-pyridin-3-yl)-imidazol-1-yl)-butylimino)]erythromycin A (Compound No. 237),

their pharmaceutically acceptable salts, pharmaceutically acceptable solvates, stereoisomers, prodrugs or polymorphs.

In another aspect, there are provided pharmaceutical compositions, which may be administered to an animal for treatment orally, topically, rectally, internasally, or by a parenteral route. The pharmaceutical compositions of the compounds provided comprise a pharmaceutically effective amount of such compound, formulated together with one or more pharmaceutically acceptable carriers.

Solid form preparation for oral administration includes capsules, tablet, pills, powder, granules, cachets and suppositories. For solid form preparation, the active compound is mixed with at least one inert, pharmaceutically acceptable excipients or carrier, for example, sodium citrate, dicalcium phosphate and/or a filler or extenders, for example, starches, lactose, sucrose, glucose, mannitol and silicic acid; binders, for example, carboxymethylcellulose, alginates, gelatins, polyvinylpyrrolidinone, sucrose, and acacia; disintegrating agents, for example, agar-agar, calcium carbonate, potato starch, alginic acid, certain silicates and sodium carbonate; absorption accelerators, for example, quaternary ammonium compounds; wetting agents, for example, cetyl alcohol, and glycerol mono stearate; adsorbants, for example, Kaolin; lubricants, for example, talc, calcium stearate, magnesium stearate, solid polyethyleneglycol, sodium lauryl sulphate and mixtures thereof. In the case of capsules, tablets, pills, the dosage form may also comprise bufferring agents.

The solid preparation of tablets, capsules, pills and granules can be prepared with coating and shells, for example, enteric coating and other coatings well known in the pharmaceutical formulating art.

Liquid form preparations for oral administration includes pharmaceutically acceptable emulsions, solutions, suspensions, syrups and elixirs. For liquid form preparations, the active compound is mixed with water or other solvent, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil), glycerol, and fatty acid esters of sorbitan and mixture thereof. Besides inert diluents, the oral composition can also include adjuants, for example, wetting agents, emulsifying agents, suspending agents, sweetening agents, flavouring agents and perfuming agents.

Injectable preparations, for example, sterile injections, aqueous suspensions may be formulated according to the art using suitable dispersing or wetting and suspending agents. Among the acceptable vehicles and solvents that may be employed are water, Ringer's solution and isotonic sodium chloride.

Dosage forms for tropical or transdermal administration of a compound of the present invention include ointments, pastes, creams, lotions, gels, powders, solutions, sprays, inhalants or patches. The active compound is admixed under sterile condition with a pharmaceutically acceptable carrier and any needed preservatives or buffers as may be required. Ophthalmic formulations, eardrops, eye ointments, powder and solution are also contemplated as being within the scope of this invention.

The pharmaceutical preparation can be presented in unit dosage form. In such forms, the preparation is subdivided into unit doses containing appropriate quantities of the active component. The unit dosage form can be a packaged preparation, the package containing discrete capsules, powders, in vials or ampoules, and ointments capsule, sachet, tablet, gel, cream itself or it can be the appropriate number of any of these packaged forms. The quantity of active compound in unit dose of preparation may be varied or adjusted from less than 1 mg to several grams according to the particular application and potency of the active ingredient.

In therapeutic use as agents for treating bacterial infections, the compounds utilizing in the pharmaceutical method of this invention can be administered at an initial dosage of about 3 mg to about 40 mg per kilogram daily. The dosages, however, may be varied depending upon the requirements of the patients and the compound being employed. Determination of the proper dosage for a particular situation may be within the smaller dosages, which are less than the optimum dose. Small increments until the optimum effect under the daily dosage may be divided and administered in portion during the day if desired.

Examples set forth below demonstrate the general synthetic procedure for the preparation of representative compounds. The examples are provided to illustrate particular aspects of the disclosure and do not constrain the scope of the present invention as defined by the claims.

### EXAMPLES

### Scheme I

### Example 1

### Preparation of Compound of Formula III

To a solution of hydrochloric acid was added clarithromycin of Formula II (25 g, 33.4 mmol). The reaction mixture was neutralized with solid sodium bicarbonate and the aqueous layer was extracted with ethyl acetate. The organic layer was washed with water, brine, and dried over anhydrous sodium sulphate. The solvent was removed under reduced pressure to give the desired product. The crude product was crystallized by using ethyl acetate-hexane mixture.

### Example 2

### Preparation of Compound of Formula IV

To a solution of compound of Formula III (I equiv) in dry dichloromethane was added benzoic anhydride (2.5 equiv), triethylamine (6 equiv) and stirred at ambient temperature. The reaction was quenched by aqueous sodium bicarbonate solution. The aqueous layer was extracted with dichloromethane, washed successively with water and brine, and dried over anhydrous sodium sulphate, and then the solvent was removed under reduced pressure to give a crude product. The crude product was crystallized by using ethyl acetate-hexane mixture.

### Example 3

### Preparation of Compound of Formula V

To a solution of compound of Formula IV (1 equiv) in dry acetonitrile: dichloromethane (2:1) was added N-iodosuccinimide (2 equiv). The reaction mixture was stirred with sodiumbisulphite solution followed by stirring with sodium carbonate solution. Dichloromethane was evaporated under reduced pressure. The aqueous matter was extracted with ethyl acetate, washed successively with water and brine, and dried over anhydrous sodium sulphate, and then the solvent was removed under reduced pressure to yield a crude product. The crude product was purified by silica gel column chromatography (thoroughly neutralized with triethylamine) using 10-20% acetone in hexane.

### Example 4

### Preparation of Compound of Formula VI

To a solution of compound of Formula V (1 equiv) in acetonitrile was added solid sodium bicarbonate (5 equiv) and a reagent of Formula R³X (6 equiv) under argon at ambient temperature. The reaction mixture was diluted with ethyl acetate and washed with water followed by brine, dried over anhydrous sodium sulphate and concentrated under reduced pressure to yield a crude product. The crude product was purified by silica gel column chromatography (thoroughly neutralized with triethylamine) using 10-20% acetone in hexane.

### Example 5

### Preparation of Compound of Formula VII

To a solution of compound of Formula VI (1 equiv) in dichloromethane was added triphosgene (1.5 equiv) with stirring. Then to it was added pyridine (15 equiv) slowly. After complete addition, reaction mixture was stirred under inert atmosphere. The reaction was quenched by addition of water. The reaction mixture was diluted with dichloromethane and washed with water and brine, dried over anhydrous sodium sulphate, and concentrated under reduced pressure to afford the desired product.

### Example 6

### Preparation of Compound of Formula VIII

To a solution of a compound of Formula VII (1 equiv) in dimethylformamide was added tetramethyl guanidine (2.2 equiv) and reaction mixture was heated. The reaction mixture was cooled to an ambient temperature and water was added and extracted with ethyl acetate. Organic layer was washed with water followed by brine, dried over anhydrous sodium sulphate and concentrated under reduced pressure to obtain the desired product.

### Example 7

### Preparation of Compound of Formula IX

To a solution of compound of Formula VIII (1 equiv) in dichloromethane was added a reagent of Formula R⁵YCOOH (2.5 equiv), 4-N-dimethylaminopyridine (2.5 equiv) and N,N′-dicyclohexylcarbodiimide (2.5 equiv) was added. Pyridine (4 equiv) was added to it. The whole reaction mixture was stirred and then filtered through a celite bed. The filtrate was washed with water and brine, dried over anhydrous sodium sulphate and concentrated under reduced pressure to afford the desired product. The crude product was purified by silica gel column chromatography (thoroughly neutralized with triethylamine) using 10-20% acetone in hexane.

### Example 8

### Preparation of Compound of Formula X

To a solution of compound of Formula IX (1 equiv) in dimethylformamide:tetrahydrofuran (3:2) was added N,N′-carbonyldiimidazole (3 equiv), followed by sodium hydride in portions. The reaction was quenched by addition of ice cold water and extracted with ethyl acetate. The ethyl acetate layer was washed with water and brine, dried over anhydrous sodium sulphate and concentrated under reduced pressure to afford the desired product.

### Example 9

### Preparation of Compound of Formula XI

A compound of Formula X (1 equiv) and a compound of Formula R—W—NH₂ (3equiv) were taken in 10% water in acetonitrile and heated. The reaction mixture was cooled to an ambient temperature; acetonitrile was evaporated under reduced pressure. The resulting residue was taken in ethyl acetate, washed with water and brine, dried over anhydrous sodium sulphate, and concentrated under reduced pressure. The resulting residue was purified by column chromatography using 25-30% acetone in hexane to afford the desired product.

### Example 10

### Preparation of Compound of Formula XII

A solution of compound of Formula XI (560 mg, 0.6 mmol) in methanol was refluxed. The reaction mixture was cooled to an ambient temperature and methanol was evaporated under reduced pressure. Purification of the solid mass was done over silica gel (thoroughly neutralized triethylamine) using 30-35% acetone in hexane or 2-8% methanol in dichloromethane.

### Scheme II

### Example 11

### Preparation of Compound of Formula XIII

A solution of compound of Formula X (1 mmol) in dry dimethylformamide and hydrazine hydrate (2.0 mmol) was added to it. The reaction mixture was stirred at room temperature for about 1.5 hour. It was quenched with water and extracted in ethyl acetate. The solvent was removed under reduced pressure and the residue was redissolved in tetrahyrofuran. Potassium-t-butoxide (2.354 mol) was added to it at 0° C. and the reaction mixture was stirred for about 2 hr. It was extracted with ethyl acetate, washed with water and brine, and dried over sodium sulphate to give the desired isomer.

### Example 12

### Preparation of Compound of Formula XIV

A solution of compound of Formula XIII in methanol was heated at 70° C. for about 20 hours. The solvent was removed and the residue was purified over silica gel column to afford the product.

### Example 13

### Preparation of Compound of Formula XV

A compound of Formula XIV (1.0 mmol) and heterocyclyl alkyl aldehyde (5.0 mmol, (prepared by following the procedure given in WO 00/17218), a compound of Formula R(CH₂)ₘCHO (5.0 mmol) were dissolved in methanol. Glacial acetic acid (5.0 mmol) was added. The resulting mixture was stirred at room temperature for about 1-2 hour. Sodium cyanoborohydride (5.0 mmol) and glacial acetic and (5.0 mmol) were added to it. The mixture was stirred at room temperature for about 12 hours, solvent was removed, the reaction mixture was extacted with dichloromethane, washed with water, brine and dried over sodium sulphate. The solvent was removed under reduced pressure and the residue was purified over silica gel column to afford the product.

### Example 14

### Preparation of Compound of Formula XVI

A compound of Formula XIV (0.298 mmol) and a compound of Formula R₈ONH₂.hydrochloride (4.471 mmol) in ethanol were stirred at 80° C. for about 48 hours. The solvent was evaporated. The reaction mixture was redissolved into ethyl acetate, washed with water and brine, dried over sodium sulphate and solvent was evaporated get the product.

### Example 15

### Preparation of Compound of Formula XVII

A compound of Formula XVI (1.0 mmol) and heterocyclyl alkyl aldehyde, a compound of Formula R(CH₂)ₘCHO (5.0 mmol) (prepared by following the procedure given in WO 00/17218), were dissolved in methanol. Glacial acetic acid (5.0 mmol) was added. The resulting mixture was stirred at room temperature for about 1-2 hour. Sodium cyanoborohydride (5.0 mmol) and glacial acetic and (5.0 mmol) were added to it. The mixture was stirred at room temperature for about 12 hours, solvent was removed, the reaction mixture was extracted with dichloromethane, washed with water, brine and dried over sodium sulphate. The solvent was removed under reduced pressure and the residue was purified over silica gel column to afford the product.

### Scheme III

### Example 16

### Preparation of Compound of Formula XVIII

To a solution of compound of Formula IV (1 equiv) in dichloromethane at 0° C. was added triphosgene (1.5 equiv) with stirring. Then to it was added pyridine (15 equiv) slowly. After complete addition, reaction mixture was stirred for about 3 h at 0-5° C. the reaction was quenched by drop wise addition of water, was diluted with dichloromethane and washed with water and brine, dried over anhydrous sodium sulphate and concentrated under reduced pressure to afford the desired product.

### Example 17

### Preparation of Compound of Formula XIX

To a solution of a compound of formula XVIII (1 equiv) in dimethylformamide was added tetramethyl guanidine (2.2 equiv) and reaction mixture was heated at 90° C. for about 8 hours. The reaction mixture was cooled to an ambient temperature and water was added and extracted with ethyl acetate, the organic layer was washed with water followed by brine, dried over anhydrous sodium sulphate and concentrated under reduced pressure to obtain the desired product.

### Example 18

### Preparation of Compound of Formula XX

To a solution of compound of Formula VI (1 equiv) in dry acetonitrile: dichloromethane (2:1) cooled to 0° C., was added N-iodosuccinimide (2 equiv). The reaction mixture was stirred with sodium bisulphite solution followed by stirring with sodium carbonate solution. Dichloromethane was evaporated under reduced pressure. The aqueous residue was extracted with ethyl acetate, washed successively with water, brine, and dried over anhydrous sodium sulphate and then the solvent was removed under reduced pressure to obtain the crude product, which was purified by silica gel column chromatography (thoroughly neutralized with triethyl amine) using 10-20% acetone in hexane to give the product.

### Example 19

### Preparation of Compound of Formula VIII

To a solution of a compound of formula VII (1 equiv) in dimethylformamide was added tetramethyl guanidine (2.2 equiv) and reaction mixture was heated. The reaction mixture was cooled to an ambient temperature and water was added and extracted with ethyl acetate. The organic layer was washed with water followed by brine, dried over anhydrous sodium sulphate and concentrated under reduced pressure to obtain the desired product.

### Example 20

### Preparation of Compound of Formula IX

To a solution of compound of Formula VIII (1 equiv) in dichloromethane was added a reagent of Formula R⁵YCOOH (2.5 equiv), 4-N-dimethylaminopyridine (2.5 equiv) and N,N′-dicyclohexylcarbodiimide (2.5 equiv) was added. Pyridine (4 equiv) was added to it. The whole reaction mixture was stirred and then filtered through celite bed. The filtrate was washed with water, brine, dried over anhydrous sodium sulphate and concentrated under reduced pressure to afford the desired product. The crude product was purified by silica gel column chromatography (thoroughly neutralized with triethylamine) using 10-20% acetone in hexane.

### Example 21

### Preparation of Compound of Formula X

To a solution of compound of Formula IX (1 equiv) in dimethylformamide:tetrahydrofuran (3:2) was added N,N′-carbonyldiimidazole (3 equiv), followed by sodium hydride in portions Reaction was quenched by addition of ice cold water and extracted with ethyl acetate. The ethyl acetate layer was washed with water, brine dried over anhydrous sodium sulphate and concentrated under reduced pressure to afford the desired product.

### Example 22

### Preparation of Compound of Formula XI

A compound of Formula X (1 equiv) and a compound of Formula R—W—NH₂ (3 equiv) were taken in 10% water in acetonitrile and heated. The reaction mixture was cooled to an ambient temperature; acetonitrile was evaporated under reduced pressure. The resulting residue was taken in ethyl acetate, washed with water, brine, dried over anhydrous sodium sulphate and concentrated under reduced pressure. The resulting residue was purified by column chromatography using 25-30% acetone in hexane to afford the desired product.

### Example 23

### Preparation of compound of Formula XII

A solution of compound of Formula XI (560 mg, 0.6 mmol) in methanol was refluxed. The reaction mixture was cooled to an ambient temperature and methanol was evaporated under reduced pressure. Purification of the solid mass was done over silica gel (thoroughly neutralized triethylamine) using 30-35% acetone in hexane or 2-8% methanol in dichloromethane.

The following illustrative compounds were prepared by following the above general procedures.
Compound No. 1: 11,12-Dideoxy-3-O-decladinosyl-3-O-(2-nitrophenyl acetyl)-5-O-(3′-N-desmethyl-3-N-ethyl)-6-O-methyl-12,11-[oxycarbonyl-((3-imidazol-1-yl)-propyl)-imino]erythromycin A, MS (+ion mode): m/z 900.5 [M+1],
Compound No. 2: 11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-imidazol-1-yl)-butyl)-imino]erythromycin A, MS (+ion mode): m/z 914.6 [M+1],
Compound No. 3: 11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-((3-imidazol-1-yl)-propyl)-imino]erythromycin A, MS (+ion mode): m/z 912.6 [M+1],
Compound No. 4: 11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-(3H)-imidazo(4,5-b)-pyridin-3-yl)-butyl)-imino]erythromycin A, MS (+ion mode): m/z 966.6 [M+1],
Compound No. 5: 11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-benzimidazol-1-yl)-butyl)-imino]erythromycin A, MS (+ion mode): m/z 965.5 [M+1],
Compound No. 6: 11,12-Dideoxy-3-O-decladinosyl-3-O-(3-nitrophenyl acetyl)-5-O-(3′-N-desmethyl-3′-N-allyl)-6-O-methyl-12,11-[oxycarbonyl-((4-benzimidazol-1-yl)-butyl)-imino]erythromycin A, MS (+ion mode): m/z 976.6 [M+1],
Compound No. 7: 11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-(prop-2-en-yl)-imino]erythromycin A, MS (+ion mode): m/z 844.7 [M+1],
Compound No. 8: 11,12-Dideoxy-3-O-decladinosyl-3-O-(4-pyridyl acetyl)-5-O-(3′-N-desmethyl-3′-N-ethyl)-6-O-methyl-12,11-[oxycarbonyl-((4-imidazol-1-yl)-butyl)-imino]erythromycin A, MS (+ion mode): m/z 870.7 [M+1],
Compound No. 9: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-imidazol-1-yl)-butyl)-imino]erythromycin A, MS (+ion mode): m/z 870.6 [M+1],
Compound No. 10: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A, MS (+ion mode): m/z 920.7 [M+1],
Compound No. 11: 11,12-Dideoxy-3-O-decladinosyl-3-O-(4-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A, MS (+ion mode): m/z 920.5 [M+1],
Compound No. 12: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A, MS (+ion mode): m/z 921.6 [M+1],
Compound No. 13: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A, MS (+ion mode): m/z 921.9 [M+1],
Compound No. 14: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A, MS (+ion mode): m/z 920.8 [M+1],
Compound No. 15: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A, MS (+ion mode): m/z 919.6 [M+1],
Compound No. 16: 11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4(4-phenyl)-imidazol-1-yl)-butyl)-imino]erythromycin A, MS (+ion mode): m/z 946.8 [M+1],
Compound No. 17: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A, MS (+ion mode): m/z 921.8 [M+1],
Compound No. 18: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-indol-1-yl)-butyl)-imino]erythromycin A, MS (+ion mode): m/z 920.8 [M+1],
Compound No. 19: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A, MS (+ion mode): m/z 919.8 [M+1],
Compound No. 20: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A, MS (+ion mode): m/z 920.8 [M+1],
Compound No. 21: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A, MS (+ion mode): m/z 921.9 [M+1],
Compound No. 22: 11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A, MS (+ion mode): m/z 946.9 [M+1],
Compound No. 23: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((N¹-methyl-N¹-pyridin-4-ylmethyl)-2-aminoethyl)-imino]erythromycin A, MS (+ion mode): m/z 896.49 [M+1],
Compound No. 24: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A, MS (+ion mode): m/z 896.42 [M+1],
Compound No. 25: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((N¹-methyl-N¹-pyridin-3-ylmethyl)-2-aminoethyl)-imino]erythromycin A, MS (+ion mode): m/z 896.42 [M+1],
Compound No. 26: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A, MS (+ion mode): m/z 946.48 [M+1],
Compound No. 27: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A, MS (+ion mode): m/z 932.89 [M+1],
Compound No. 28: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A, MS (+ion mode): m/z 933.94 [M+1],
Compound No. 29: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A, MS (+ion mode): m/z 933.94 [M+1],
Compound No. 30: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-(2-methyl)-benzoimidazo-1-yl)-butyl)-imino]erythromycin A, MS (+ion mode): m/z 934.79 [M+1],
Compound No. 31: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A, MS (+ion mode): m/z 958.98 [M+1],
Compound No. 32: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-pyridin-2-yl)-butyl)-imino]erythromycin A, MS (+ion mode): m/z 881.78 [M+1],
Compound No. 33: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-pyridin-4-yl)-butyl)-imino]erythromycin A, MS (+ion mode): m/z 881.85 [M+1],
Compound No. 34: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-benzoimidazo-1-yl)-butyl)-imino]erythromycin A, MS (+ion mode): m/z 932.8 [M+1],
Compound No. 35: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A, MS (+ion mode): m/z 959.83 [M+1],
Compound No. 36: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-benzotriazol-1-yl)-butyl)-imino]erythromycin A, MS (+ion mode): m/z 933.94 [M+1],
Compound No. 37: 11,12-dideoxy-3-O-decladinosyl-3-O-(4-pyridyl acetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A, MS (+ion mode): m/z 947.96 [M+1],
Compound No. 38: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A, MS (+ion mode): m/z 947.96 [M+1],
Compound No. 39: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A, MS (+ion mode): m/z 947.96 [M+1],
Compound No. 40: 11,12-dideoxy-3-O-decladinosyl-3-O-(4-pyridyl acetyl)-5-O-(3′-N-desmethyl-3′-N-ethyl)-6-O-methyl-12,11-[oxycarbonyl-((4-pyrrolo[2,3-b]pyridin-1-yl)-butyl)-imino]erythromycin A, MS (+ion mode): m/z 920.96 [M+1],
Compound No. 41: 11,12-dideoxy-3-O-decladinosyl-3-O-(2-pyridyl acetyl)-5-O-(3′-N-desmethyl-3′-N-propargyl)-6-O-methyl-12,11-[oxycarbonyl-((4-(3H)imidazo[4,5-b]pyridin-3-yl)-butyl)-imino]erythromycin A, MS (+ion mode): m/z 931.94 [M+1],
Compound No. 42: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A, MS (+ion mode): m/z 933.99 [M+1],
Compound No. 43: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((9-(4-amino-butyl)9H-purin-6-yl)-imino]erythromycin A, MS (+ion mode): m/z 949.83 [M+1],
Compound No. 44: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-benzoimidazo-1-yl)-butyl)-imino]erythromycin A, MS (+ion mode): m/z 933.01 [M+1],
Compound No. 45: 11,12-dideoxy-3-O-decladinosyl-3-O-(4-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A, MS (+ion mode): m/z 946.81 [M+1],
Compound No. 46: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A, MS (+ion mode): m/z 958.82 [M+1],
Compound No. 47: 11,12-dideoxy-3-O-decladinosyl-3-O-(4-pyridyl acetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A, MS (+ion mode): m/z 921.83 [M+1],
Compound No. 48: 11,12-dideoxy-3-O-decladinosyl-3-O-(4-pyridyl acetyl)-5-O-(3′-N-desmethyl-3′-N-ethyl)-6-O-methyl-12,11-[oxycarbonyl-((4-benzotriazol-1-yl)-butyl)-imino]erythromycin A, MS (+ion mode): m/z 921.59 [M+1],
Compound No. 49: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A, MS (+ion mode): m/z 959.92 [M+1],
Compound No. 50: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A, MS (+ion mode): m/z 932.66 [M+1],
Compound No. 51: 11,12-dideoxy-3-O-decladinosyl-3-O-(2-pyridyl acetyl)-5-O-(3′-N-desmethyl-3′-N-propargyl)-6-O-methyl-12,11-[oxycarbonyl-((4-benzoimidazo-1-yl)-butyl)-imino]erythromycin A, MS (+ion mode): m/z 930.64 [M+1],
Compound No. 52: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-benzotriazol-1-yl)-butyl)-imino]erythromycin A, MS (+ion mode): m/z 921.65 [M+1],
Compound No. 53: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A, MS (+ion mode): m/z 933.63 [M+1],
Compound No. 54: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-benzotriazol-1-yl)-butyl)-imino]erythromycin A, MS (+ion mode): m/z 933.7[M+1],
Compound No. 55: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-benzotriazol-1-yl)-butyl)-imino]erythromycin A, MS (+ion mode): m/z 921.71 [M+1],
Compound No. 56: 11,12-dideoxy-3-O-decladinosyl-3-O-(2-pyridyl acetyl)-5-O-(3′-N-desmethyl-3′-N-propargyl)-6-O-methyl-12,11-[oxycarbonyl-((4-pyrrolo[2,3-b]pyridin-1-yl)-butyl)-imino]erythromycin A, MS (+ion mode): m/z 930.91 [M+1],
Compound No. 57: 11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-furan-2-yl)-imidazol-1-yl)-butyl)-imino]erythromycin A, MS (+ion mode): m/z 948.42 [M+1],
Compound No. 58: 11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-thiophen-2-yl)-imidazol-1-yl)-butyl)-imino]erythromycin A, MS (+ion mode): m/z 964.33 [M+1],
Compound No. 59: 11,12-dideoxy-3-O-decladinosyl-3-O-(2-pyridylacetyl)-5-O-(3′-N-desmethyl-3′-N-allyl)-6-O-methyl-12,11-[oxycarbonyl-((9-(3-hydrazino-propyl)-9-H-purin-6-yl-4-amino)]erythromycin A, MS (+ion mode): m/z 950.39 [M+1],
Compound No. 60: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(quinolin-8-yl)-butyl)-imino)]erythromycin A, MS (+ion mode): m/z 931.44 [M+1],
Compound No. 61: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-8-yl)-butyl)-imino)]erythromycin A, MS (+ion mode): m/z 943.40 [M+1],
Compound No. 62: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-4-yl)-butyl)-imino)]erythromycin A, MS (+ion mode): m/z 943.44 [M+1],
Compound No. 63: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1-methyl-1H-pyrrolo[2,3-b]pyridin-3-yl)-butyl)-imino)]erythromycin A, MS (+ion mode): m/z 946.86 [M+1],
Compound No. 64: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-amino-9H-purin-9-yl butylimino)]erythromycin A, MS (+ion mode): m/z 949.62 [M+1],
Compound No. 65: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A, MS (+ion mode): m/z 937.62 [M+1],
Compound No. 66: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A, MS (+ion mode): m/z 943.62 [M+1],
Compound No. 67: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A, MS (+ion mode): m/z 931.59 [M+1],
Compound No. 68: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A, MS (+ion mode): m/z 964.59 [M+1],
Compound No. 69: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A hydrochloride salt,
Compound No. 70: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A, MS (+ion mode): m/z 977.65 [M+1],
Compound No. 71: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(6-pyrrol-1-yl-purin-9-yl)-butyl)-imino)]erythromycin A, MS (+ion mode): m/z 999.64 [M+1],
Compound No. 72: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-diethylamino-9H-purin-9-yl butylimino)]erythromycin A, MS (+ion mode): m/z 1005.62 [M+1],
Compound No. 73: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-ethylamino-9H-purin-9-yl butylimino)]erythromycin A, MS (+ion mode): m/z 978.72 [M+1],
Compound No. 74: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A, MS (+ion mode): m/z 965.43 [M+1],
Compound No. 75: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A, MS (+ion mode): m/z 977.45 [M+1],
Compound No. 76: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A, MS (+ion mode): m/z 963.33 [M+1],
Compound No. 77: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A, MS (+ion mode): m/z 963.39 [M+1],
Compound No. 78: 11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A, MS (+ion mode): m/z 939.30 [M+1],
Compound No. 79: 11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A, MS (+ion mode): m/z 939.37 [M+1],
Compound No. 80: 11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A, MS (+ion mode): m/z 955.30 [M+1],
Compound No. 81: 11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A, MS (+ion mode): m/z 955.39 [M+1],
Compound No. 82: 11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A, MS (+ion mode): m/z 939.38 [M+1],
Compound No. 83: 11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A, MS (+ion mode): m/z 939.38 [M+1],
Compound No. 84: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A, MS (+ion mode): m/z 965.39 [M+1],
Compound No. 85: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-methylamino-9H-purin-9-yl butylimino)]erythromycin A, MS (+ion mode): m/z 965.37 [M+1],
Compound No. 86: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(purin-9-yl)-butyl)-imino)]erythromycin A, MS (+ion mode): m/z 934.35 [M+1],
Compound No. 87: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(purin-9-yl)-butyl)-imino)]erythromycin A, MS (+ion mode): m/z 934.34 [M+1],
Compound No. 88: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-c]pyridin-1-yl)-butyl)-imino)]erythromycin A, MS (+ion mode): m/z 933.37 [M+1],
Compound No. 89: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(3H-imidazo[4,5-c]pyridin-3-yl)-butyl)-imino)]erythromycin A, MS (+ion mode): m/z 933.35 [M+1],
Compound No. 90: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-1yl)-butyl)-imino)]erythromycin A, MS (+ion mode): m/z 964.40 [M+1],
Compound No. 91: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A, MS (+ion mode): m/z 933.35 [M+1],
Compound No. 92: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazol[4,5-c]pyridin-3-yl)-butyl)-imino)]erythromycin A, MS (+ion mode): m/z 933.44 [M+1],
Compound No. 93: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A, MS (+ion mode): m/z 943.39 [M+1],
Compound No. 94: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-oxazol-5-yl-1H-imidazol-1yl)-butyl)-butyl)-imino)]erythromycin A, MS (+ion mode): m/z 949.62 [M+1],
Compound No. 95: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A, MS (+ion mode): m/z 948.67 [M+1],
Compound No. 96: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A hydrochloride salt,
Compound No. 97: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A, MS (+ion mode): m/z 948.67 [M+1],
Compound No. 98: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A, MS (+ion mode): m/z 948.53 [M+1],
Compound No. 99: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-pyrazol-1yl)-butyl)-imino)]erythromycin A, MS (+ion mode): m/z 964.56 [M+1],
Compound No. 100: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-pyrazol-1yl)-butyl)-imino)]erythromycin A, MS (+ion mode): m/z 948.73 [M+1],
Compound No. 101: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-2-yl-1H-pyrazol-1yl)-butyl)-imino)]erythromycin A, MS (+ion mode): m/z 948.57 [M+1],
Compound No. 102: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-thiophen-2-yl-1H-pyrazol-1yl)-butyl)-imino)]erythromycin A, MS (+ion mode): m/z 964.70 [M+1],
Compound No. 103: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A, MS (+ion mode): m/z 937.50 [M+1],
Compound No. 104: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-(quinolin-3-yl)-butyl)-imino)]erythromycin A, MS (+ion mode): m/z 931.50 [M+1],
Compound No. 105: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-3-yl)-butyl)-imino)]erythromycin A, MS (+ion mode): m/z 943.50 [M+1],
Compound No. 106: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-([1,4′]bipyrazolyl-1′-yl)-butyl)-imino)]erythromycin A, MS (+ion mode): m/z 948.77 [M+1],
Compound No. 107: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiazol-2-yl-1H-imidazol-1-yl)-butyl)-imino)]erythromycin A, MS (+ion mode): m/z 965.68 [M+1],
Compound No. 108: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-thiazol-2-yl-1H-imidazol-1-yl)-butyl)-imino)]erythromycin A, MS (+ion mode): m/z 965.68 [M+1],
Compound No. 109: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-pyrazol-1yl)-imino)]erythromycin A, MS (+ion mode): m/z 948.73 [M+1],
Compound No. 110: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-pyrazol-1yl)-butyl)-imino)]erythromycin A, MS (+ion mode): m/z 964.77 [M+1],
Compound No. 111: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-thiophen-3-yl-1H-pyrazol-1yl)-butyl)-imino)]erythromycin A, MS (+ion mode): m/z 964.81 [M+1],
Compound No. 112: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-pyrazol-1yl)-butyl)-imino)]erythromycin A, MS (+ion mode): m/z 948.83 [M+1],
Compound No. 113: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A, MS (+ion mode): m/z 960.44 [M+1],
Compound No. 114: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-benzoimidazol-1-yl)-propyl)-hydrazo)]erythromycin A, MS (+ion mode): m/z 933.41 [M+1],
Compound No. 115: (R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1yl)-propyl)-hydrazo)]erythromycin A, MS (+ion mode): m/z 960.63 [M+1],
Compound No. 116: (S or R) 11,12-dideoxy-3-O-decladinosyl-3-O-(2-pyridyl acetyl)-5O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A, MS (+ion mode): m/z 960.64 [M+1],
Compound No. 117: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-(3-(4-pyridin-3-yl-1H-imidazol-1-yl)-propyl)-hydrazo)]erythromycin A, MS (+ion mode): m/z 948.72 [M+1],
Compound No. 118: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-pyridin-3-yl-1H-imidazol-1yl)-propyl)-hydrazo)]erythromycin A, MS (+ion mode): m/z 960.24 [M+1],
Compound No. 119: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(4-pyridin-3-yl-1H-imidazol-1yl)-propyl)-hydrazo)]erythromycin A, MS (+ion mode): m/z 948.25 [M+1],
Compound No. 120: 11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(3-(6-amino-9H-purin-9-yl-propyl)hydrazo)]erythromycin A, MS (+ion mode): m/z 938.36 [M+1],
Compound No. 121: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1yl)-propyl)-hydrazo)]erythromycin A, MS (+ion mode): m/z 965.39 [M+1],
Compound No. 122: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A, MS (+ion mode): m/z 944.54 [M+1],
Compound No. 123: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A, MS (+ion mode): m/z 935.49 [M+1],
Compound No. 124: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A, MS (+ion mode): m/z 935.49 [M+1],
Compound No. 125: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-2-yl)-1H-imidazol-1-yl)propyl)-hydrazo)]erythromycin A, MS (+ion mode): m/z 949.54 [M+1],
Compound No. 126: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A, MS (+ion mode): m/z 950.70 [M+1],
Compound No. 127: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A, MS (+ion mode): m/z 949.62 [M+1],
Compound No. 128: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A, MS (+ion mode): m/z 949.66 [M+1],
Compound No. 129: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A, MS (+ion mode): m/z 950.67 [M+1],
Compound No. 130: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A, MS (+ion mode): m/z 965.57 [M+1],
Compound No. 131: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A, MS (+ion mode): m/z 965.63 [M+1],
Compound No. 132: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-3-yl-1H-imidazol-1-yl)-propyl)-hydrazo)]erythromycin A, MS (+ion mode): m/z 965.58 [M+1],
Compound No. 133: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A, MS (+ion mode): m/z 934.65 [M+1],
Compound No. 134: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A, MS (+ion mode): m/z 934.79 [M+1],
Compound No. 135: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A, MS (+ion mode): m/z 966.66 [M+1],
Compound No. 136: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A, MS (+ion mode): m/z 966.66 [M+1],
Compound No. 137: (R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A, MS (+ion mode): m/z 775.52 [M+1],
Compound No. 138: (S or R) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A, MS (+ion mode): m/z 775.52 [M+1],
Compound No. 139: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-hydrazo)]erythromycin A, MS (+ion mode): m/z 763.59 [M+1],
Compound No. 140: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A, 9-(O-methyl)oxime, MS (+ion mode): m/z 989.68 [M+1],
Compound No. 141: 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A, 9-(O-methyl)oxime, MS (+ion mode): m/z 804.58 [M+1],
Compound No. 142: 11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-(6-fluoro-pyridin-3-yl)-1H-imidazol-1-yl)-butyl)-imino)]erythromycin A, MS (+ion mode): m/z 977.63 [M+1],
Compound No. 143: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(6-fluoro-pyridin-3-yl)-1H-imidazol-1-yl)-butyl)-imino)]erythromycin A, MS (+ion mode): m/z 977.40 [M+1],
Compound No. 144: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(1H-imidazol-1-yl)phenyl)-butyl)-imino)]erythromycin A, MS (+ion mode): m/z 958.64 [M+1],
Compound No. 145: 11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(5-(3-aminophenyl)-thiazol-2-yl)-butyl)-imino)]erythromycin A, MS (+ion mode): m/z 960.50 [M+1],
Compound No. 146: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-amino-pyrimidin)-5-yl)-imidazol-1-yl)-butylimino)]erythromycin A, MS (+ion mode): m/z 975.5 [M+1],
Compound No. 147: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-aminopyridin-4-yl)-imidazol-1-yl)-butylimino)]erythromycin A, MS (+ion mode): m/z 974.6 [M+1],
Compound No. 148: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-aminopyridin-3-yl)-imidazol-1-yl)-butylimino)]erythromycin A, MS (+ion mode): m/z 974.6 [M+1],
Compound No. 49: 11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-(dimethylamino)-9H-purin-9-yl)-butylimino)]erythromycin A, MS (+ion mode): m/z 965.39 [M+1],
Compound No. 150: 11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(3-(5-phenyl-tetrazol-1-yl)propylimino)]erythromycin A, MS (+ion mode): m/z 990.4 [M+1],
Compound No. 151: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyridin-3-yl-pyrazol-1-yl)-butylimino)]erythromycin A, MS (+ion mode): m/z 975.64 [M+1],
Compound No. 152: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methyl-2H-tetrazol-5-yl)phenyl)butylimino)]erythromycin A, MS (+ion mode): m/z 974.51 [M+1],
Compound No. 153: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(3-thienyl)-imidazol-1-yl)butylimino)]erythromycin A, MS (+ion mode): m/z 964.56 [M+1],
Compound No. 154: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(5-methyl-[1,3,4]-oxadiazol-2-yl)phenyl)butylimino)]erythromycin A, MS (+ion mode): m/z 974.47 [M+1],
Compound No. 155: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([2,3′]-bithiophenyl-5-yl)butylimino)]erythromycin A, MS (+ion mode): m/z 980.50 [M+1],
Compound No. 156: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([2,3′]-bithiophenyl5-yl)butylimino)]erythromycin A, MS (+ion mode): m/z 980.60 [M+1],
Compound No. 157: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A, MS (+ion mode): m/z 980.40 [M+1],
Compound No. 158: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A, MS (+ion mode): m/z 980.40 [M+1],
Compound No. 159: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A, MS (+ion mode): m/z 961.50 [M+1],
Compound No. 160: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-imidazol-1-yl)butylimino)]erythromycin A, MS (+ion mode): m/z 948.60 [M+1],
Compound No. 161: 11,12-dideoxy-3-O-decladinosyl-3-O-(2-methylpropanoyl)-5-O-(3′-N-desmethyl-3′-N-allyl)-6-O-methyl-12,11-[oxycarbonyl4-(4-(4-(furan-2-yl)-imidazol-1-yl)-butylimino)]erythromycin A, MS (+ion mode): m/z 899.63 [M+1],
Compound No. 162: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(4-methoxy-phenyl)-imidazol-1-yl)-butylimino)]erythromycin A, MS (+ion mode): m/z 988.5 [M+1],
Compound No. 163: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(4-methoxy-phenyl)-imidazol-1-yl)-butylimino)]erythromycin A, MS (+ion mode): m/z 988.50 [M+1],
Compound No. 164: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A, MS (+ion mode): m/z 988.50 [M+1],
Compound No. 165: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H -imidazol-1-yl)-butylimino)]erythromycin A, MS (+ion mode): m/z 988.50 [M+1],
Compound No. 166: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A, MS (+ion mode): m/z 974.60 [M+1],
Compound No. 167: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A, MS (+ion mode): m/z 974.60 [M+1],
Compound No. 168: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2 -yl)butylimino)]erythromycin A, MS (+ion mode): m/z 964.40 [M+1],
Compound No. 169: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A, MS (+ion mode): m/z 964.40 [M+1],
Compound No. 170: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A, MS (+ion mode): m/z 990.50 [M+1],
Compound No. 171: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A, MS (+ion mode): m/z 990.50 [M+1],
Compound No. 172: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A, MS (+ion mode): m/z 960.50 [M+1],
Compound No. 173: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A, MS (+ion mode): m/z 961.50 [M+1],
Compound No. 174: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A, MS (+ion mode): m/z 961.50 [M+1],
Compound No. 175: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A, MS (+ion mode): m/z 960.50 [M+1],
Compound No. 176: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl)butylimino)]erythromycin A, MS (+ion mode): m/z 993.50 [M+1],
Compound No. 177: 11,12-dideoxy-3-O-decladinosyl-3-O-(2-pyridyl acetyl)-5-O-(3′-N-desmethyl-3′-N-allyl)-6-O-methyl-12,11-[oxycarbonyl-3 ((3-(4-(imidazol-1-yl)-pyrazol-1-yl)-propyl)-hydrazo)]erythromycin A, MS (+ion mode): m/z 949.50 [M+1],
Compound No. 178: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrazol-1-yl]-imidazol-1-yl)-propyl)-hydrazo)]erythromycin A, MS (+ion mode): m/z 950.40 [M+1],
Compound No. 179: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A, MS (+ion mode): m/z 973.40 [M+1],
Compound No. 180: 11,12-dideoxy-3-O-decladinosyl-3-O-(3-pyridyl acetyl)-5-O-(3′-N-desmethyl-3′-N-allyl)-6-O-methyl-12,11-[oxycarbonyl-3-((3-([1,4′]-bipyrazol-1′-yl)-propyl)-hydrazo)]erythromycin A, MS (+ion mode): m/z 949.70 [M+1],
Compound No. 181: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A, MS (+ion mode): m/z 937.70 [M+1],
Compound No. 182: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A, MS (+ion mode): m/z 960.50 [M+1],
Compound No. 183: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrimidin-2-yl-imidazol-1-yl)-propyl)-hydrazo]erythromycin A, MS (+ion mode): m/z 961.50 [M+1],
Compound No. 184: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A, MS (+ion mode): m/z 960.50 [M+1],
Compound No. 185: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A, MS (+ion mode): m/z 950.60 [M+1],
Compound No. 186: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(thiophen-2-yl)-tetrazol-2-yl])-butylimino)]erythromycin A, MS (+ion mode): m/z 966.50 [M+1],
Compound No. 187: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-1-yl]-butylimino)]erythromycin A, MS (+ion mode): m/z 972.50 [M+1],
Compound No. 188: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-1-yl]-butylimino)]erythromycin A, MS (+ion mode): m/z 972.50 [M+1],
Compound No. 189: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A, MS (+ion mode): m/z 950.60 [M+1],
Compound No. 190: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-3-yl-tetrazol-2-yl)-butylimino)]erythromycin A, MS (+ion mode): m/z 961.50 [M+1],
Compound No. 191: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A, MS (+ion mode): m/z 961.40 [M+1],
Compound No. 192: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A, MS (+ion mode): m/z 961.40 [M+1],
Compound No. 193: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A, MS (+ion mode): m/z 966.50 [M+1],
Compound No. 194: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A, MS (+ion mode): m/z 966.50 [M+1],
Compound No. 195: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(thiophen-2-yl-tetrazol-2-yl)-butylimino)]erythromycin A, MS (+ion mode): m/z 966.50 [M+1],
Compound No. 196: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A, MS (+ion mode): m/z 932.57 [M+1],
Compound No. 197: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A, MS (+ion mode): m/z 934.00 [M+1],
Compound No. 198: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A, MS (+ion mode): m/z 934.05 [M+1],
Compound No. 199: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A, MS (+ion mode): m/z 950.58 [M+1],
Compound No. 200: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A, MS (+ion mode): m/z 950.52 [M+1],
Compound No. 201: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A, MS (+ion mode): m/z 932.70 [M+1],
Compound No. 202: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A, MS (+ion mode): m/z 978.6 [M+1],
Compound No. 203: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A, MS (+ion mode): m/z 978.48 [M+1],
Compound No. 204: 11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(benzimidazol-1-yl)-butylimino)]erythromycin A, MS (+ion mode): m/z 883.62 [M+1],
Compound No. 205: 11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-(3-pyridyl)-imidazol-1-yl)-butylimino)]erythromycin A, MS (+ion mode): m/z 932.75 [M+1],
Compound No. 206: 11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A, MS (+ion mode): m/z 884.45 [M+1],
Compound No. 207: 11,12-dideoxy-3-O-decladinosyl-3-O-(2-methyl propanoyl)-5-O-(3′-N-desmethyl-3′-N-allyl)-6-O-methyl-12,11-[oxycarbonyl-4-(6-amino-9H-purin-9-yl)butylimino)]erythromycin A, MS (+ion mode): m/z 901.33 [M+1],
Compound No. 208: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-bipyrazol-1′-yl)butylimino]erythromycin A, MS (+ion mode): m/z 948.56 [M+1],
Compound No. 209: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A, MS (+ion mode): m/z 948.38 [M+1],
Compound No. 210: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A, MS (+ion mode): m/z 948.56 [M+1],
Compound No. 211: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-biimidazol-1′-yl)butylimino)]erythromycin A, MS (+ion mode): m/z 949.06 [M+1],
Compound No. 212: 11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A, MS (+ion mode): m/z 983.46 [M+1],
Compound No. 213: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A, MS (+ion mode): m/z 948.38 [M+1],
Compound No. 214: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A, MS (+ion mode): m/z 933.60 [M+1],
Compound No. 215: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A, MS (+ion mode): m/z 933.50 [M+1],
Compound No. 216: 11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(4-(6-fluoro-pyridin-3-yl)-imidazol-1-yl)butylimino)]erythromycin A, MS (+ion mode): m/z 928.5 [M+1],
Compound No. 217: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A, MS (+ion mode): m/z 975.6 [M+1],
Compound No. 218: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A, MS (+ion mode): m/z 975.6 [M+1],
Compound No. 219: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-((3-(4-pyrimidin-2-yl)-imidazol-2-yl)propyl)-hydrazo)]erythromycin A, MS (+ion mode): m/z 961.6 [M+1],
Compound No. 220: 11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(4-pyrimidin-5-yl)-imidazol-2-yl)propyl)-hydrazo)]erythromycin A, MS (+ion mode): m/z 961.55 [M+1],
Compound No. 221: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A, MS (+ion mode): m/z 956.50 [M+1],
Compound No. 222: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A, MS (+ion mode): m/z 959.5 [M+1],
Compound No. 223: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-phenyl)butylimino)]erythromycin A, MS (+ion mode): m/z 970.6 [M+1],
Compound no. 224: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrimidin-5-yl-imidazol-1-yl)propyl)hydrazo)]erythromycin A, MS (+ion mode): m/z 961.54 [M+1],
Compound No. 225: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(imidazol-1-yl)-pyrazol-1-yl]propyl}hydrazo)]erythromycin A, MS (+ion mode): m/z 949.5 [M+1],
Compound No. 226: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-{[3-([1,4′]-biimidazol-1′-yl)propyl}hydrazo)]erythromycin A, MS (+ion mode): m/z 949.7 [M+1],
Compound No. 227: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A, MS (+ion mode): m/z 949.5 [M+1],
Compound No. 228: 11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A, MS (+ion mode): m/z 934.5 [M+1],
Compound No. 229: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A, MS (+ion mode): m/z 949.5 [M+1],
Compound No. 230: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A, MS (+ion mode): m/z 948.50 [M+1],
Compound No. 231: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A, MS (+ion mode): m/z 948.40 [M+1],
Compound No. 232: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2H-tetrazol-5-yl)-phenyl)butylimino)]erythromycin A, MS (+ion mode): m/z 960.49 [M+1],
Compound No. 233: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(2H-tetrazol-5-yl)-phenyl]butylimino)]erythromycin A, MS (+ion mode): m/z 960.49 [M+1],
Compound No. 234: 11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A, MS (+ion mode): m/z 993.50 [M+1],
Compound No. 235: 11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A, MS (+ion mode): m/z 950.40 [M+1],
Compound No. 236: 11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A, MS (+ion mode): m/z 976.6 [M+1],
Compound No. 237: 11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-fluoro-pyridin-3-yl)-imidazol-1-yl)-butylimino)]erythromycin A, MS (+ion mode): m/z 977.40 [M+1].

### Example 24

### Pharmacological Activity

Compounds provided herein displayed antibacterial activity in vitro, especially against strains which are generally resistant to macrolides either due to efflux (mef strains) or ribosomal modification (erm) strains. These compounds are useful in the treatment of community-acquired pneumonia, upper- and lower-respiratory tract infections, skin and soft tissue infections, hospital-acquired lung infections, bone and joint infections, and other bacterial infections, for example, mastitis, catether infection, foreign body, prosthesis infections or peptic ulcer disease. Minimum inhibitory concentration (MIC) has been an indicator of in vitro antibacterial activity widely used in the art.

### Procedure

### Medium

a) Cation adjusted Mueller Hinton Agar (MHA-Difco)
b) Trypticase Soya Agar (TSA)

### Inoculum Preparation

The cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well-isolated colonies were taken and saline suspensions were prepared in sterile densimat tubes. The turbidity of the culture was adjusted to 0.5-0.7 Mc Farland standard (1.5×10⁸ CFU/ml). The cultures were diluted 10 fold in saline to get inoculum size of approximately 1-2×10⁷ organisms/ml.

### Preparation of Drug Concentration

1 mg/ml concentration of stock solution of drugs was prepared in dimethylsulfoxide/distilled water/solvent given in National Committee for Clinical Laboratory Standards (NCCLS) manual. Serial two-fold dilutions of the compounds and standard drugs were prepared as per NCCLS manual. Stock solution was changed according to the need of the experiment.

### Preparation of Agar Plates

Two ml of respective drug concentration was added to 18 ml of Molten Mueller Hinton agar to get the required range, for example 0.015 μg/ml-16 μg/ml. For fastidious cultures, 1 ml of sheep blood was added in Molten Mueller Hinton agar. For control, MHA and MHA with 5% sheep blood plates without antibiotic for each set were prepared. One MHA and MHA with 5% sheep blood plate without antibiotic for determining quality check for media was prepared.

### Preparation of Teflon Template

1 μl of each culture on each plate was replicated with the help of replicator (Denley's multipoint replicator). The spots were allowed to dry and the plates were incubated for about 18-24 hours at 37° C. Fastidious cultures were incubated at 37° C. in CO₂ incubator. The results were noted comparing with the control plates.

### Endpoint Definition

The concentration of drug at which there was complete disappearance of growth spot or formation of less than 10 colonies per spot was considered as Minimum Inhibitory Concentration (MIC). The MICs of Quality Control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

### Staphylococcus aureus ATCC 29213

### Enterococcus faecalis ATCC 29212

### Eschericia coli ATCC 25922

### Pseudomonas aeruginosa ATCC 27853

All 60 cultures were visually checked for purity.

Media Control: NCCLS disc diffusion assay using 10 μg discs of Gentamicin (Difco) against Pseudomonas aeruginosa ATCC 27853. A zone diameter of 16-21 mm was considered for optimum cation (Magnesium and Calcium) content of the media. The diameter was plotted in the media QC chart.

### REFERENCES

National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-S12, Vol. 22 No. 1 (January 2002).

Results: compounds disclosed herein have shown activity against Staphylococcus aureus, Streptococcus pneumoniae, Haemophilus influenzae, Moraxella catarrhalis Streptococcus pyogenes, Enterococci faecalis, Enterococci faecium and Helicobacter pylori.

The results of the antibacterial activity of the compounds disclosed herein are as follows:
a) The compounds disclosed herein exhibited MIC values against Staphylococcus aureus (25923) in the range of between about 0.03 μg/mL and about 16 μg/mL, for example, between about 0.03 μg/mL and about 4 μg/mL, or between about 0.03 μg/mL and about 0.25 μg/mL or between about 0.03 μg/mL and about 0.125 μg/mL.
b) The compounds disclosed herein exhibited MIC values against sensitive Streptococcus pneumoniae (6303, 49619) in the range of between about 0.008 μg/mL to about 16 μg/mL, for example between about 0.008 μg/mL to about 0.125 μg/mL, or between about 0.008 μg/mL to about 0.06 μg/mL.
c) The compounds disclosed herein exhibited MIC values against erythromycin-resistant Streptococcus pneumoniae (AB14 erm, AB29 erm, 1275 erm, AB34 mef, CS 1687 mef, 3579, 3390, 4745, 994, 5055, or 5051) in the range of between about 0.008 μg/mL to about 16 μg/mL, for example between about 0.008 μg/mL to about 1 μg/mL, or between about 0.008 μg/mL to about 0.125 μg/mL, or between about 0.008 μg/mL to about 0.03 μg/mL.
d) The compounds disclosed herein exhibited MIC values against telithromycin-resistant Streptococcus pneumonia in the range of between about 0.25 μg/mL to about 16 μg/mL, between about 0.25 μg/mL to about 4 μg/mL, or between about 0.25 μg/mL to about 1 μg/mL.
e) The compounds disclosed herein exhibited MIC values against Haemophilus influenzae (49247, 38) in the range of between about 0.03 μg/mL to about 16 μg/mL, for example between about 0.03 μg/mL to about 2 μg/mL, or from between about 0.03 μg/mL to about 0.125 μg/mL.
f) The compounds disclosed herein exhibited MIC values against Moraxella catarrhalis (8176, M6) in the range of between about 0.015 μg/mL to about 16 μg/mL, for example, between about 0.015 μg/mL to about 1 μg/mL, or from between about 0.015 μg/mL to about 0.06 μg/mL.
g) The compounds disclosed herein exhibited MIC values against sensitive Streptococcus pyogenes (19615) in the range of between about 0.008 μg/mL to about 2 μg/mL, for example, between about 0.008 μg/mL to about 0.125 μg/mL, or from about 0.008 μg/mL to about 0.06 μg/mL.
h) The compounds disclosed herein exhibited MIC values against erythromycin-resistant Streptococcus pyogenes (1721 emb, 2534 erm TR) in the range of between about 0.004 μg/mL to about 16 μg/mL, for example, between about 0.004 μg/mL to about 0.125 μg/mL, or between about 0.004 μg/mL to about 0.03 μg/mL.
i) The compounds disclosed herein exhibited MIC values against Helicobacter pylori (43504) in the range of between about 0.03 μg/mL to about 1 μg/mL.
j) The compounds disclosed herein exhibited MIC values against sensitive E. faecalis (29212) in the range of between about 0.03 μg/mL to about 4 μg/mL, for example from between about 0.03 μg/mL to about 0.25 μg/mL, or from between about 0.03 μg/mL to about 0.125 μg/mL.
k) The compounds disclosed herein exhibited MIC values against Vancomycin-resistant Enterococci (346, 6A) in the range of between about 0.125 μg/mL to about 16 μg/mL, for example, between about 0.125 μg/mL to about 4 μg/mL, or from about between about 0.125 μg/mL to about 0.5 μg/mL.

We claim:

1. Compounds having the structure of Formula I, 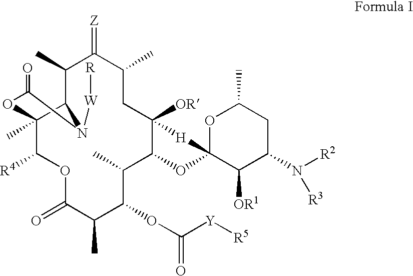
pharmaceutically acceptable salts, pharmaceutically acceptable solvates, stereoisomers, prodrugs or polymorphs thereof, wherein:
R¹ is hydrogen or a hydroxyl protecting group;
R² and R³ are independently alkyl, alkenyl, alkynyl, cycloalkyl, aryl, heterocycle, aralkyl or (heterocycle)alkyl (with the proviso that R² and R³ simultaneously are not methyl);
R⁴ is alkyl, alkenyl or alkynyl;
R⁵ is alkyl, aryl or heterocycle;
R is no atom, hydrogen, aryl or heterocycle;
R′ is alkyl or —(CH₂)_{q}—U—V (wherein q is an integer of from 1 to 4, U is alkenyl or alkynyl, V is hydrogen, aryl or heterocycle);
W is alkenyl, -G(CH₂)ₘJ, —CR⁹R¹⁰, —NR⁹— or —SO₂— {[wherein m is an integer of from 2 to 6, G is no atom, —CO, —CS, —SO₂ or —NR⁹, J is no atom or —N((R⁹)(CH₂)ₙ— (wherein n is an integer of from 1 to 4, R⁹ and R¹⁰ are independently hydrogen or alkyl)]}; Y is -Q(CH₂)ₖ—, {wherein k is an integer of from 1 to 6, Q is no atom, —NR⁹— or oxygen [wherein R⁹ is hydrogen or alkyl]}, further alkylene chain of -Q(CH₂)ₖ— is optionally substituted with alkyl, hydroxy or alkoxy; and
Z is oxygen, sulphur or NOR⁸ (wherein R⁸ is hydrogen, alkyl or aralkyl).

2. The compound according to claim 1, wherein:
R¹ is hydrogen; R is no atom or heterocycle; R² and R³ are independently alkyl, alkenyl or alkynyl (with the proviso that R² and R³ simultaneously are not methyl); R⁴ is ethyl; R⁵ is alkyl, aryl or heterocycle; R′ and R⁴ are alkyl; W is alkenyl or -G(CH₂)ₘJ-; Z is oxygen or sulphur; Y is -Q(CH₂)ₖ, wherein G, J, Q, m and k are the same as defined in claim 1.

3. A compound, which is selected from:
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((3-imidazol-1-yl)-propyl)-imino]erythromycin A,
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-imidazol-1-yl)-butyl)-imino]erythromycin A,
11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-((3-imidazol-1-yl)-propyl)-imino]erythromycin A,
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-(3H)-imidazo(4,5-b)-pyridin-3-yl)-butyl)-imino]erythromycin A,
11,12-Dideoxy-3-O-decladinosyl-3-O-(2-nitrophenyl acetyl)-5-O-(3′-N-desmethyl-3′-N-ethyl)-6-O-methyl-12,11-[oxycarbonyl-((4-benzimidazol-1-yl)-butyl-imino]erythromycin A,
11,12-Dideoxy-3-O-decladinosyl-3-O-(3-nitrophenyl acetyl)-5-O-(3′-N-desmethyl-3′-N-allyl)-6-O-methyl-12,11-[oxycarbonyl-((4-benzimidazol-1-yl)-butyl)-imino]erythromycin A,
11,12-Dideoxy-3-O-decladinosyl-3-O-(2-nitrophenyl acetyl)-5-O-(3′-N-desmethyl-3′-N-allyl)-6-O-methyl-12,11-[oxycarbonyl-(prop-2-en-yl)-imino]erythromycin A,
11,12-Dideoxy-3-O-decladinosyl-3-O-(4-pyridyl acetyl)-5-O-(3′-N-desmethyl-3′-N-ethyl)-6-O-methyl-12,11-[oxycarbonyl-((4-imidazol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-imidazol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A,
11,12-Dideoxy-3-O-decladinosyl-3-O(4-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-indol-1-yl)-butyl)-imino]erythromycin A,
11,12-Dideoxy-3-O-decladinosyl-3-O-(2-pyridyl acetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A,
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(1H)-imidazol-[4,5-b]pyridin-1-yl)-butyl)-imino]erythromycin A,
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-indol-1-yl)-butyl)-imino]erythromycin A,
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-benzimidazol-1-yl)-butyl)-imino]erythromycin A,
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-pyrrolo-[2,3-b]pyridin-1-yl)-butyl)-imino]erythromycin A,
11,12-Dideoxy-3-O-decladinosyl-3-O-(3-pyridyl acetyl)-5-O-(3′-N-desmethyl-3′-N-ethyl)-6-O-methyl-12,11-[oxycarbonyl-((4-(3H)-imidazol-[4,5-b]pyridin-3-yl)-butyl)-imino]erythromycin A,
11,12-Dideoxy-3-O-decladinosyl-3-O-(3-pyridylacetyl-5-O-(3′-N-desmethyl-3′-N-ethyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-2-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-pyridin-3-ylmethyl)-2-aminoethyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((N¹-methyl-N¹-quinolin-4-ylmethyl)-2-aminoethyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-(2-methyl)-benzoimidazo-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-pyridin-2-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-pyridin-4-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-benzoimidazo-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-benzotriazol-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-(4-pyridin-3-yl)-imidazol-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-pyrrolo[2,3-b]pyridin-1-yl)-butyl)-imino]erythromycin A,
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((9-(4-amino-butyl)9H-purin-6-yl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-benzoimidazo-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-phenyl)-imidazol-1-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-(3H)imidazo[4,5-b]pyridin-3-yl)-butyl)-imino]erythromycin A,
11,12-dideoxy-3-O-decladinosyl-3-O-(4-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-pyridin-3-yl)-imidazol-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-pyrrolo[2,3-b]pyridin-1-yl)-butyl)-imino]erythromycin A,
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-benzoimidazo-1-yl)-butyl)-imino]erythromycin A,
11,12-dideoxy-3-O-decladinosyl-3-O-(2-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(1H)imidazo[4,5-b]pyridin-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-benzotriazol-1-yl)-butyl)-imino]erythromycin A,
11,12-dideoxy-3-O-decladinosyl-3-O-(3-pyridylacetyl)-5-O-(3′-N-desmethyl-3′-N-ethyl)-6-O-methyl-12,11-[oxycarbonyl-((4-benzotriazol-1-yl)-butyl)-imino]erythromycin A,
11,12-dideoxy-3-O-decladinosyl-3-O-(2-pyridylacetyl)-5-O-(3′-N-desmethyl-3′-N-propargyl)-6-O-methyl-12,11-[oxycarbonyl-((4-pyrrolo[2,3-b]pyridin-1-yl)-butyl)-imino]erythromycin A,
11,12-dideoxy-3-O-decladinosyl-3-O-(2-pyridylacetyl)-5-O-(3′-N-desmethyl-3′-N-allyl)-6-O-methyl-12,11-[oxycarbonyl-((4-(4-furan-2-yl)-imidazol-1-yl)-butyl)-imino]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-((4-(4-thiophen-2-yl)-imidazol-1-yl)-butyl)-imino]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3[4-(6-amino-9H-purin-9-yl)propyl)hydrazo)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(quinolin-8-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-8-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-4-yl)-butyl)-imino]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1-methyl-1H-pyrrolo[2,3-b]pyridin-3-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-amino-9H-purin-9-yl butylimino)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-amino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-((4-(isoquinolin-5-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-3-yl-1H-imidazol-1-yl)-butyl)-imino)]erythromycin A hydrochloride salt,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(6-pyrrol-1-yl-purin-9-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-diethylamino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-ethylamino-9H-purin-9-yl butylimino)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-dimethylamino-9H-purin-9-yl butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(6-methylamino-9H-purin-9-yl butylimino)]erythromycin A,
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A,
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A,
11,12-dideoxy-3-O-decladinosyl-3-O-(2-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A,
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-(4-(6-amino-9H-purin-9-yl butylimino)]erythromycin A,
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(1H-imidazo[4,5-b]pyridin-1-yl)-butyl)-imino)]erythromycin A,
11,12-dideoxy-3-O-decladinosyl-3-O-(3-pyridyl acetyl)-5-O-(3′-N-desmethyl-3′-N-2-fluoroethyl)-6-O-methyl-12,11-[oxycarbonyl-((4-(3H-imidazo[4,5-b]pyridin-3-yl)-butyl)-imino)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-dimethylamino-9H-purin-9-yl butylimino)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-methylamino-9H-purin-9-yl butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(purin-9-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(purin-9-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(1H-imidazo[4,5-c]pyridin-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(3H-imidazo[4,5-c]pyridin-3-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-thiophen-2-yl-1H-imidazol-1-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(1H-imidazol[4,5-c]pyridin-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(3H-imidazol[4,5-c]pyridin-3-yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(isoquinolin-5-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-oxazol-5-yl-1H-imidazol-1yl)-butyl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-2-yl-1H-imidazol-1yl)-butyl)-imino)]erythromycin A hydrochloride salt,
11,12-dideoxy-3-O-decladinosyl-3-O-(3-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-furan-3-yl-1H-imidazol-1yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-2-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-2-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-2-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-2-yl)-1H-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(6-amino-9H-purin-9-yl butylimino)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-((4-(quinolin-3-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(quinolin-3-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-4-([1,4′]-bipyrazol-1′-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-thiazolyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiazol-2-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(thiophen-3-yl)-pyrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(furan-3-yl)-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-benzoimidazol-1-yl)-propyl)-hydrazo)]erythromycin A,
(R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1yl)-propyl)-hydrazo)]erythromycin A,
(S or R) 11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(3-(4-pyridin-3-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-pyridin-3-yl-1H-imidazol-1yl)-propyl)-hydrazo)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(4-pyridin-3-yl-1H-imidazol-1yl)-propyl)-hydrazo)]erythromycin A,
11,12-dideoxy-3-O-decladinosyl-3-O-(2-pyridyl acetyl)-5-O-(3′-N-desmethyl-3′-N-ethyl)-6-O-methyl-12,11-[oxycarbonyl-(3-(3-(6-amino-9H-purin-9-yl-propyl)hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiophen-3-yl)-1H-imidazol-1yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-isoquinolin-5-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-purin-9-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-purin-9-yl)-propyl)-hydrazo)]erythromycin A,
11,12-dideoxy-3-O-decladinosyl-3-O-(2-pyridyl acetyl)-5-O-(3′-N-dimethyl-3′-N-allyl)-6-O-methyl-12,11-[oxycarbonyl-(3-(4-(furan-2-yl)-1H-imidazol-1-yl)propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A,
11,12-dideoxy-3-O-decladinosyl-3-O-(3-pyridyl acetyl)-5-O-(3′-N-desmethyl-3′-N-allyl-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(furan-3-yl)-1H-imidazol-1-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(6-amino-9H-purin-9-yl-propyl)hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4″-(thiophen-2-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-2-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiophen-3-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(imidazo[4,5-b]pyridin-3-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(4-(thiazol-2-yl-1H-imidazol-1-yl)-propyl)-hydrazo)]erythromycin A,
(R or S) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A,
(S or R) 11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((3-(4-pyridin-3-yl-1H-imidazol-1-yl)-propyl)-hydrazo)]erythromycin A, 9-(O-methyl)oxime,
11,12-dideoxy-3-O-decladinosyl-3-O-(2-pyridyl acetyl)-5-O-(3′-N-desmethyl-3′-N-allyl)-6-O-methyl-12,11-[oxycarbonyl-hydrazo]erythromycin A, 9-(O-methyl)oxime,
11,12-dideoxy-3-O-decladinosyl-3-O-(3-pyridyl acetyl)-5-O-(3′-N-desmethyl-3′-N-allyl)-6-O-methyl -12,11-[oxycarbonyl-((4-(4-(6-fluoro-pyridin-3-yl) -1H-imidazol-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(6-fluoro-pyridin-3-yl)-1H-imidazol-1-yl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(4-(1H-imidazol-1-yl)phenyl)-butyl)-imino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-((4-(5-(3-aminophenyl)-thiazol-2-yl)-butyl)-imino)]erythromycin A, 11,12-dideoxy-3-O-decladinosyl-3-(2-pyridyl acetyl)-5-O-(3′-N-desmethyl-3′-N-allyl)-6-O-methyl-12,11-[oxycarbonyl-(4-(4-(2-amino-pyrimidin)-5-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-aminopyridin-4-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl -(4-(4-(6-aminopyridin-3-yl)-imidazol-1-yl)-butylimino)]erythromycin A,
11,12-dideoxy-3-O-decladinosyl-3-O-(3-pyridyl acetyl)-5-O-(3′-N-desmethyl-3′-N-ethyl)-6-O-methyl-12,11-[oxycarbonyl-(4-(6-(dimethylamino)-9H-purin-9-yl)-butylimino)]erythromycin A,
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(3-(5-phenyl-tetrazol-1-yl)propylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyridin-3-yl-pyrazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methyl-2H-tetrazol-5-yl)phenyl)butylimino)]erythromycin A,
11,12-dideoxy-3-O-decladinosyl-3-O-(3-pyridyl acetyl)-5-O-(3′-N-desmethyl-3 ′-N-allyl)-6-O-methyl-12,11-[oxycarbonyl-(4-(4-(3-thienyl)-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(5-methyl-[1,3,4]-oxadiazol-2-yl)phenyl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([2,3′]-bithiophenyl-5-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([2,3′]-bithiophenyl-5-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[3,3′]-bithiophenyl-5-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-3-yl)-tetrazol-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-imidazol-1-yl)butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-4-(4-(4-(furan-2-yl)-imidazol-1-yl)-butylimino)]erythromycin A,
11,12-dideoxy-3-O-decladinosyl-3-O-(2-pyridyl acetyl)-5-O-(3′-N-desmethyl-3 ′-N-allyl)-6-O-methyl-12,11-[oxycarbonyl-(4-(4-(4-methoxy-phenyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(4-methoxy-phenyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(2-methoxy-phenyl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(2-methoxy-phenyl)-1H-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-phenyl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-furan-2-yl-thiophen-2-yl)butylimino)]erythromycin A,
11,12-dideoxy-3-O-decladinosyl-3-O-(2-pyridy acetyl)-5-O-(3′-N-desmethyl-3′-N-allyl)-6-O-methyl-12,11-[oxycarbonyl-(4-(4-furan-2-yl-thiophen-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(4-methoxyphenyl)-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-4-yl-tetrazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-phenyl-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3((3-(4-(imidazol-1-yl)-pyrazol-1-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-(3-(4-pyrazol-1-yl]-imidazol-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-3-((3-([1,4′]-bipyrazol-1′-yl)-propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(3-aminophenyl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrimidin-2-yl-imidazol-1-yl)-propyl)-hydrazo]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(thiophen-2-yl)-tetrazol-2-yl])-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-(furan-2-yl)-tetrazol-1-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-1-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(furan-2-yl)-tetrazol-2-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-3-yl-tetrazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-pyridin-2-yl-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A,
11,12-dideoxy-3-O-decladinosyl-3-O-(3-pyridylacetyl)-5-O-(3′-N-desmethyl-3′-N-allyl)-6-O-methyl-12,11-[oxycarbonyl-(4-(5-thiophen-2-yl-tetrazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(5-(thiophen-2-yl-tetrazol-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(1H-imidazol[4,5-b]pyridin-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-tetrazol-1-yl)-imidazol-1-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(1H-benzimidazol-2-yl)-butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-({3-[4-(6-fluoro-pyridin-3-yl-imidazol-1-yl]propyl}hydrazo]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(benzimidazol-1-yl)-butylimino)]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-(3-pyridyl)-imidazol-1-yl)-butylimino)]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(4-imidazol[4,5-b]pyridin-1-yl)-butylimino)]erythromycin A,
11,12-dideoxy-3-O-decladinosyl-3-O-(2-methyl propanoyl)-5-O-(3′-N-desmethyl-3′-N-allyl)-6-O-methyl-12,11-[oxycarbonyl-(4-(6-amino-9H-purin-9-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([1,4′]-bipyrazol-1′-yl)butylimino]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(pyrazol-1-yl)-imidazol-1-yl)butylimino]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-([1,4′]-biimidazol-1′-yl) butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-([14′]-biimidazol-1′-yl)butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(imidazo[4,5-b]pyridin-3-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-pyrazol-1-yl)-imidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(1H-imidazo[4,5-c]pyridin-2-yl)butylimino)]erythromycin A,
11,12-dideoxy-3-O-decladinosyl-3-O-(2-methylpropanoyl)-5-O-(3′-N-desmethyl-3′-N-allyl)-6-O-methyl-12,11-[oxycarbonyl-(4-(4-(6-fluoro-pyridin-3-yl)-imidazol-1-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A,
11,12-dideoxy-3-(3-decladinosyl-3-O-(2-pyridyl acetyl)-5-O-(3′-N-desmethyl-3′-N-allyl)-6-O-methyl-12,11-[oxycarbonyl-(4-(4-phenyl-thiazol-2-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-((3-(4-pyrimidin-2-yl)-imidazol-2-yl)propyl)-hydrazo)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(4-pyrimidin-5-yl)-imidazol-2-yl)propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(1H-[1,2,4]-triazol-1-yl)phenyl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-pyrimidin-5-yl-phenyl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(3-(3-(4-pyrimidin-5-yl-imidazol-1-yl)propyl)hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-({3-[4-(imidazol-1-yl)-pyrazol-1-yl]propyl}hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-{[3-([1,4′]-biimidazol-1′-yl)propyl}hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(3-(3-(4-pyrazol-1-yl)-imidazol-1-yl)-propyl)-hydrazo]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-(1H-imidazo[4,5-b]pyridine-1-yl)-propyl)-hydrazo)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(oxazol-5-yl)-imidazol-1-yl)butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-imidazol-1-yl)-pyrazol-1-yl]butylimino)]erythromycin A,
11,12-dideoxy-3-O-decladinosyl-3-O-(3-pyridyl acetyl)-5-O-(3′-N-desmethyl-3′-N-allyl)-6-O-methyl -12,11-[oxycarbonyl-(4-(4-(2H-tetrazol-5-yl)-phenyl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-[4-(2H-tetrazol-5-yl)-phenyl]butylimino)]erythromycin A,
11,12-dideoxy-3-O-decladinosyl-3-O-(3-pyridyl acetyl)-5-O-(3′-N-desmethyl-3′-N-allyl)-6-O-methyl-12,11-[oxycarbonyl-(4-(4-(6-chloropyridin-3-yl)-imidazol-1-yl]butylimino)]erythromycin A.
11,12-dideoxy-3-O-decladinosyl-3-O-(2-pyridyl acetyl)-5-O-(3′-N-desmethyl-3′-N-allyl)-6-O-methyl-12,11-[oxycarbonyl-3-((3-([1,4′]-bipyrazol-1-yl)-)-propyl)-hydrazo)]erythromycin A,
11,12-dideoxy-3-O-decladinosyl-3-O-(2-nitrophenyl acetyl)-5-O-(3′-N-desmethyl-3′-N-allyl)-6-O-methyl-12,11-[oxycarbonyl-(4-benzimidazol-1-yl)butylimino)]erythromycin A,
11,12-dideoxy-3-O-decladinosyl-3-O-(2-pyridyl acetyl)-5-O-(3′-N-desmethyl-3′-N-allyl)-6-O-methyl-12,11-[oxycarbonyl-(4-(4-(6-fluoro-pyridin-3-yl)-imidazol-1-yl)-butylimino)]erythromycin A, and
their pharmaceutically acceptable salts, pharmaceutically acceptable solvates, stereoisomers, prodrugs or polymorphs.

4. A pharmaceutical composition comprising a therapeutically effective amount of a compound of claim 1, together with at least one pharmaceutically acceptable carrier, excipient or diluent.

5. A method for treating or preventing a mammal suffering from a condition caused by or contributed to by bacterial infection, comprising administering to the mammal a therapeutically effective amount of a compound of claim 1.

6. A method for treating or preventing a mammal suffering from a condition caused by or contributed to by bacterial infection, comprising administering to the mammal a therapeutically effective amount of a pharmaceutical composition of claim 4.

7. The method according to claim 5 wherein the condition is selected from the group consisting of community acquired pneumonia, upper and lower respiratory tract infections, skin and soft tissue infections, hospital acquired lung infections or bone and joint infections, and other bacterial infections, for example, mastitis, catether infection, foreign body or prosthesis infections.

8. The method according to claim 5 wherein the bacterium is Gram-positive, Gram-negative or anaerobic bacteria.

9. The method according to claim 8 wherein bacterium is selected from the group comprising of Staphylococci, Streptococci, Enterococci, Haemophilus, Moraxalla spp., Chlamydia spp., Mycoplasm, Legionella spp., Mycobacterium, Helicobacter, Clostridium, Bacteroides, Cornebacterium, Bacillus and Enterobactericeae.

10. The method according to claim 9 wherein the bacterium is cocci.

11. The method according to claim 10 wherein the cocci is drug resistant.

12. A process for preparing a compound of Formula XII, 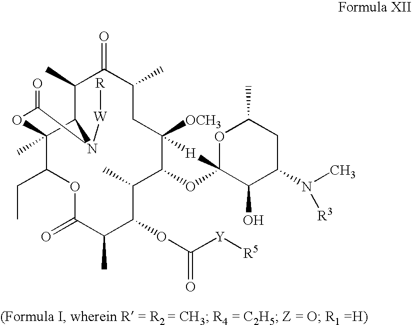
and its pharmaceutically acceptable salts, pharmaceutically acceptable solvates, stereoisomers, prodrugs or polymorphs, wherein:
R³, R⁵, R, W and Y are the same as defined in claim 1, which comprises:
(a) hydrolyzing clarithromycin of Formula II, 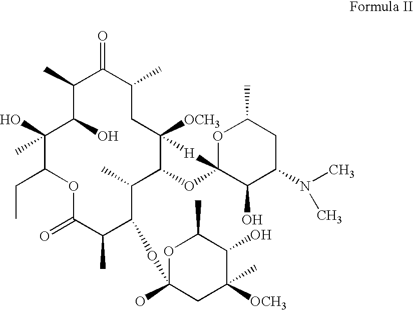
to give a compound of Formula III, 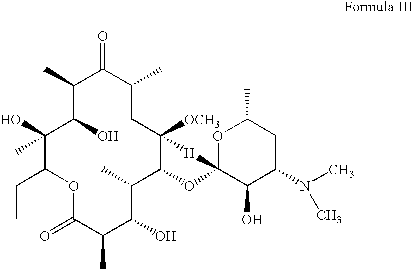
(b) the compound of Formula III is protected with a reagent of Formula R¹₂O or R¹X (wherein X is halogen) to give a compound of Formula IV (wherein R¹ is —COPh), 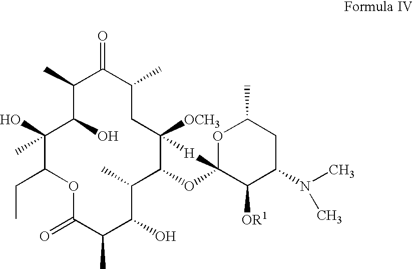
(c) the compound of Formula IV is desmethylation at 3′-N-dimethyl to give a compound of Formula V, 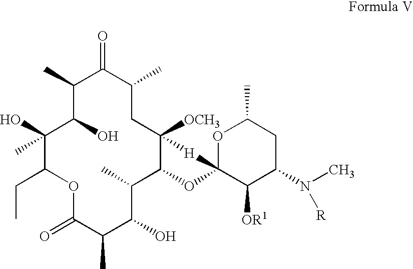
(d) the compound of Formula V is alkylated with a reagent of Formula R³CHO, R³₂CO or R³X (wherein X is halogen) to give a compound of Formula VI (wherein RJ is the same as defined earlier), 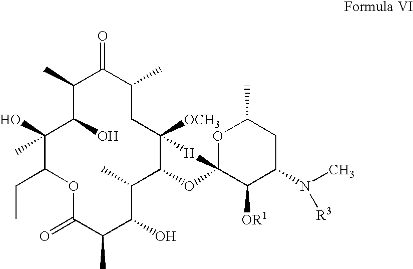
(e) the compound of Formula VI is reacted with a suitable reagent to give a compound of Formula VII, 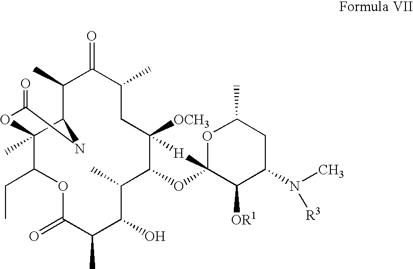
(f) the compound of Formula VII is reacted with a suitable base to give a compound of Formula VIII, 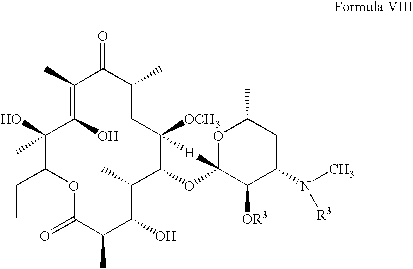
(g) the compound of Formula VIII is reacted with a compound of Formula R⁵YCOOH, (R⁵YCO)₂O, R⁵YCOX or R⁵YCOOR¹⁰ (wherein R¹⁰ is a leaving group) to give a compound of Formula IX (wherein Y and R⁵ are the same as defined earlier), 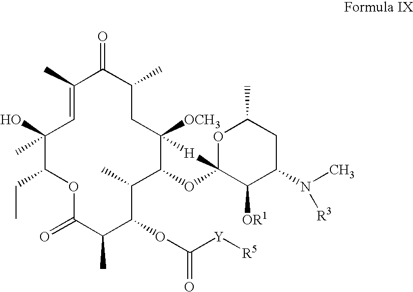
(h) the compound of Formula IX is reacted with N,N′-carbonyl diimidazole to give a compound of Formula X, 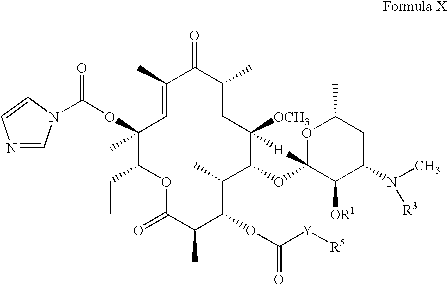
(i) the compound of Formula X is reacted with a compound of Formula R—W—NH₂ to give a compound of Formula XI (wherein W and R are the same as defined earlier), 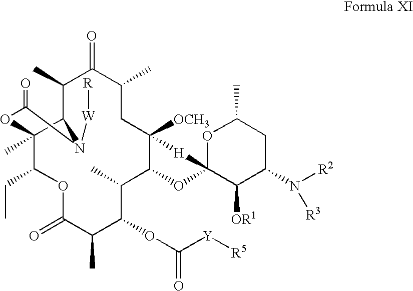
(j) the compound of Formula XI is finally deprotected to give a compound of Formula XII.

13. (canceled)

14. (canceled)

15. (canceled)
